(19) United States
(12) Patent Application Publication
(10) Pub. No.: US 2021/0406841 A1
(43) Pub. Date: Dec. 30, 2021

(54) VIRTUAL VISIT OBJECTS

(71) Applicant: DRFIRST.COM, INC., Rockville, MD (US)

(72) Inventors: James F. Chen, Naples, FL (US); Linda Fischer, Oakdale, NY (US)

(21) Appl. No.: 17/247,472

(22) Filed: Dec. 11, 2020

Related U.S. Application Data
(60) Provisional application No. 62/705,483, filed on Jun. 29, 2020.

(51) Int. Cl. G06Q 10/10; G16H 10/60; G16H 40/20; H04N 7/15

(52) U.S. Cl. CPC G06Q 10/1095; G16H 10/60; H04L 51/046; H04N 7/15; G16H 40/20

(57) ABSTRACT

Systems and methods provide an infrastructure for supporting wholly or partially virtual visits. Example methods include instantiating a virtual visit object for a subject participant, assigning a virtual room in the virtual visit object to a first room status, assigning at least a service team member to the virtual room based on room access rules applicable to the first room status. The method may also include providing a first user interface to the subject participant configured according to room participant access rules for the first room status to collect a data element from the subject participant. Changing the room status to a second room status may change the user interface, add a second service team member to the room participants for the virtual room based on a second room access rule applicable to the second room status and may remove the first service team member from the room participants.

### REFERENCE TO RELATED APPLICATION

This application is a non-provisional of, and claims priority to, U.S. Provisional Application No. 62/705,483, filed Jun. 29, 2020, the disclosure of which is incorporated herein by reference.

### BACKGROUND

Telemedicine visits enable a patient to speak with a medical provider without having to physically visit an office. Use of telemedicine by patients and physicians has been growing but has been limited to phone calls and hangouts that exist independently of existing medical enterprise systems.

### SUMMARY

Disclosed systems and methods provide technical solutions to technical problems presented by virtual interactions or virtual visits to an institution or an organization, such as, for example, a telemedicine visit to a healthcare institution, a virtual interview with a potential employer, or a virtual class or a virtual school day in an education institution, etc. In the physical world, a physician providing care in a healthcare institution has on-site resources and follows a specific intake and post-visit procedure set up by the healthcare institution. The on-site resources help organize patient care in a flow of care events in various settings and provide sharing of collection of information related to a visit. However, such event flow organization and information sharing among various care events are unavailable in a telemedicine visit. Similarly, in an on-site interview, a recruiter or office manager leads an interviewee through the physical process, escorting the interviewee to the different offices or conference rooms where different interviews take place. Further, in a school day, a student may take multiple classes throughout the day, each class is given in a specific room, with a specific teacher and a specific group of students. Implementations provide a digital infrastructure for organizing and managing a virtual visit that may consist of one or more virtual sessions, such as a waiting room session, an exam room session during a visit for medical care, a virtual interview session during an interview visit, a virtual class or a virtual campus tour during a virtual school visit, etc. The infrastructure can integrate with existing enterprise systems to define the flow of virtual events in each virtual session. The digital infrastructure can provide confidentiality of a subject (a patient, an interviewee, a student), including the subject's personal information, such as, for example, health information related to a subject patient, employment history information for a job candidate interviewee, education or class performance information for a student, etc. The digital infrastructure can facilitate controlled sharing of information related to the subject, and can provide an event history related to the virtual visit, etc. Implementations also take advantage of functionality not available via traditional settings. The subject participant of the virtual visit is a person to whom the host enterprise/organization/institution provides services and around whom the one or many virtual sessions/events are organized, e.g., the patient, the potential job candidate, the student, etc.

The infrastructure includes a virtual visit object generated for a subject participant. A virtual visit object includes a virtual room for the virtual service (e.g., one or more sessions of medical care or other type of visit for the subject). The virtual room has a room status. The virtual room status reflects the current purpose of the virtual room, or in other words the purpose of the current virtual session. The room status can change over time reflecting different purposes or different sessions. For example, the virtual room can have a pre-exam or pre-admission status (similar to a waiting room), an active exam status, a post-op status, an imaging status, a pre-op status, a recovery status, etc. In comparison to a physical visit, where a subject participant moves from one physical room to another, the virtual room changes its room status to reflect the concept of physical room changes of the subject participant. The room status of the virtual room may be associated with the purpose of the room. The status of the virtual room may also be associated with a name or an address of the room.

The virtual room may have room participants, or in other words, specific people or people of specific roles that are associated with the purpose of the room. The room participants, which includes the subject participant, are enabled to engage in virtual interactions among them, via one or more communication channels. In one disclosed implementation, one or more of the room participants are determined based on the room status as defined by enterprise rules, also referred to as room access rules. In some implementations, the rules relating to respective participants' level of access may be referred to as room participant access rules. For example, room participant access rules may govern who has access to the virtual room, which communication channel is accessible to each respective room participant, and their respective level of access, based on, for example, the room status and the room participant role, etc. In some implementations, the room participant access rule also governs what data from an enterprise system the room participants can have access to. In some implementations, the room access rules may also govern what actions may be available for a subject participant or a room participant to take, etc., for example, what forms might be available for completion/submission. The room access rule may also include what data is collected from a participant in a room, and what data is available to whom for access within the virtual visit object, rules on what data is permitted for import and export with an external records system.

The virtual visit object may also include a separate communication channel for a service team, such as, for example, members, agents or delegates of the host/enterprise entrusted to provide services to the subject participant, but does not include the subject participant. The separate service team communication channel may include, for example, a subject-centered chat (e.g., Backline® patient centered chat by DrFirst®), or an internal communication channel of the host/enterprise, and optionally multiple secure side communication channels between members of the service team. Members of the service team may have access to the virtual room, such as, for example, to communicate with the subject participant in real-time, based on the room status.

The enterprise rules may include one set of team access rules for determining members of the service team (team participants) and another set of rules for determining who has access to the virtual room (room access rules), both of which may be based on room status. In some implementations, team access rules may include team participant access rules, which define a level of access for the team and/or a particular team member. Likewise, in some implementations, room access rules may include room participant access rules, which define a level of access in the room and/or for a particular room member. Implementations provide an infrastructure that encapsulates the data and services to make it easier for a subject participant and other participants (e.g., other room participants or team participants) to participate in, conduct, or manage a virtual visit. The virtual visit object also helps to provide a complete picture for the virtual visit, such as providing records of one or more events during each service session that occurred in the virtual visit. The virtual visit object thus provides continuity that facilitates transitions in the team participants, such as, for example, enabling a member of the service team to enter and exit the virtual room, and also providing an event history for new service team members. Implementations also enable a medical institution/enterprise to provide the subject patient with seamless access to services (menus for service requests, identification of or contacts to nursing team, etc.), including enabling such access to people the subject patient invites to the room, sometimes based on a room status. Implementations thus describe an infrastructure that facilitates collaboration, organizes workflow and controlled sharing of information, and integrates with existing enterprise systems to provide secure interfaces automatically customized to the subject participant, the stage of the visit, and the hosting enterprise.

In a medical implementation, the virtual visit object can be used with or without telemedicine (e.g., at least one telephone or video conference between a patient and medical professional). For example, implementations can provide role-specific dashboards based on the virtual room status that help a patient, including the patient's selected representatives, communicate with service team members (e.g., a doctor, a nurse or a staff) during inpatient or outpatient visits. Implementations can provide role-specific interfaces for team members appropriate for the room status. Implementations can also be used in other environments, such as a corporate or education environment. Implementations enable providers (e.g., HR managers, physician's offices, hospitals, educational institutions) to offer a subject patient/interviewee/student access to personnel data, such as menus, schedules, test results, etc., during a virtual visit, such as an session of care, an interview, an orientation, a virtual class, etc. Thus, the virtual visit object can be used in any virtual or partially virtual visits, e.g. for virtual or partially-virtual job candidate interviews, personal virtual campus tours, virtual sales presentations, etc.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 describes a high-level depiction of a virtual visit object environment, according to a disclosed implementation.

FIG. 2 illustrates a depiction of additional elements in a virtual visit object environment, according to a disclosed implementation.

FIG. 3 illustrates a flowchart of an example process for creating a virtual visit object, according to a disclosed implementation.

FIG. 4 illustrates flowchart of an example process for providing a virtual room, according to a disclosed implementation.

FIG. 5 illustrates a first example user interface for a physician initiating a virtual visit, according to a disclosed implementation.

FIG. 6 illustrates an example user interface for a patient in the virtual room with a ‘waiting room’ status, according to a disclosed implementation.

FIG. 7 illustrates an example user interface for a physician in a virtual room with an ‘exam room’ status, according to a disclosed implementation.

FIG. 8 illustrates an example user interface for a patient in a virtual room with an ‘exam room’ status, according to a disclosed implementation.

FIG. 9 illustrates an example user interface for a secure video session that occurs in the virtual room with an ‘exam room’ status, according to a disclosed implementation.

FIG. 10 illustrates an example user interface for a secure video session that occurs in the virtual room with an ‘exam room’ status, according to a disclosed implementation.

FIG. 11 illustrates an example user interface for a physician in the virtual room with a ‘post exam room’ status, according to a disclosed implementation.

FIG. 12 illustrates an example user interface for a physician in the virtual room with a ‘post exam room’ status, according to a disclosed implementation.

FIG. 13 illustrates an example user interface for a patient in the virtual room with a ‘post exam room’ status subsequent to the secure video session, according to a disclosed implementation.

FIG. 14 illustrates an example user interface for a patient in the virtual room with a ‘recovery room’ status, according to a disclosed implementation.

### DETAILED DESCRIPTION

Implementations provide an infrastructure, referred to as a virtual visit object, to organize, document, and facilitate communications regarding a virtual visit. The virtual visit object is centered around the subject participant and implements a virtual room that moves with the subject participant during the visit. In other words, the virtual room changes purpose (e.g., room status) during the virtual visit to simulate, for example, a location change of the subject patient as compared to a physical visit. The virtual room change dictates the change of the other participants, either in the room with the subject or in the service team for the subject. The virtual room change also dictates data and communications changes according to the room status. The infrastructure also provides a separate communication space where other visit participants can communicate about the subject participant where the subject patient is not enabled to access. Implementations provide customizable access rules, which programmatically determine access to the two communication channels of the virtual object (e.g., to a room channel that includes the subject participant; to a subject-centered channel that excludes the subject participant) based on room status. An example visit is an episode of care for a medical visit. The visit may be wholly virtual or may be a physical visit with virtual components. In either case, the virtual visit object enables those that cannot be/choose not to be physically present with the subject participant to participate via the virtual visit, communicate regarding the care, etc.

Disclosed implementations provide a system that creates a virtual visit object for each virtual visit to a host institution, such as a medical care provider, a medical institution, a university, a school, a business, etc. A virtual visit may also be referred to as a service episode which includes one or more service sessions. Each service session may describe a time period during the service episode, such as, for example, a time period when the subject is in a particular room and/or a room has a particular status. Thus, a service session may also be defined by the room status of the virtual room during the virtual visit. In one example, a medical virtual visit includes a waiting room session, an exam room session, a discharging room session. In another example, a virtual interview visit includes a reception session, and one or more interviewing sessions. A service session may be associated to a room address if the service session relates to or assimilates a physical visit. In one implementation, a service session includes all events during the time period when the subject was serviced in the single physical room or all events during the time period when the room has a particular room status.

The virtual visit may begin at the request of a subject entity (e.g., a patient requesting a virtual office visit, upon admittance to an emergency room, request of a campus visit, etc.). A virtual visit may begin in response to an action taken by the enterprise (e.g. scheduling a medical virtual visit request, scheduling of a virtual job interview, scheduling of orientation, scheduling one or more classes etc.) The virtual visit ends a predetermined time after the virtual visit ends. The predetermined time may be dependent on the type of virtual service. Thus, for example, a virtual visit object related to an outpatient hospital visit may extend for a period of time, e.g., two days, a week, three weeks, etc., after the physical visit to the hospital, whereas a virtual visit object for a virtual visit object related to a telehealth visit or job interview may extend five minutes, an hour, a few hours, a day, etc. In some implementations, the system may delete virtual visit objects older than the predetermined time period. In some implementations, the end of the visit may occur when the room status changes to a post-visit status, e.g., ‘post exam room,’ discharge room,' exit room,' etc. The predetermined time may be set by access rules set by an enterprise representing the care provider (e.g., hospital rules, physician's office rules, registrar's rules, etc.). As used herein, an enterprise is any organization (e.g., hospital, physician's office, human resources department, student services department, etc.) that coordinates the visit and determines the customizable settings, etc. for the system that provides the virtual visit objects.

A virtual visit object encapsulates and organizes the data and personnel involved in the visit, e.g., an episode of medical care, a virtual orientation, a virtual school day, etc. A virtual visit object is generated for a subject participant. A subject participant can be any person for whom the visit is being coordinated (e.g., a patient, a job candidate, a potential customer or student, a tourist, a matriculated student, etc.) The subject participant may also be described as having (or being assigned to) a subject participant role for the virtual visit object. A virtual visit object is associated with a virtual room. The virtual room has a room status. The room status indicates the current purpose of the virtual room. The purpose of the virtual room may change throughout the visit. The status of a virtual room is used to identify access rules. Access rules are rules set by the enterprise for the visit that define who has access to a virtual visit object, what information is available in the interfaces for a virtual visit object, what information is collected for a virtual visit object and when, what level of access a participant has, etc. Thus, access rules can define who is invited to a virtual room based on room status (room access rules), can define who is a service team participant based on room status (team access rules), can identify information collected from one or more participants in a virtual room and/or can identify what information is displayed to a particular participant (room participant access rules), etc. Some access rules can apply to the virtual object as a whole. For instance, some personnel may be associated with a particular virtual visit object because of their role and the subject participant to which the virtual visit object applies. For example, a physician may be assigned to a virtual visit object (e.g., as a team participant and/or a room participant) because the patient for that virtual visit object is a patient of the physician. Likewise, a recruiter may be assigned to a virtual visit object because the recruiter is assigned to the job candidate for which the virtual visit is generated. Thus, no matter what the room status is, the physician or recruiter has access to the virtual visit object. Put another way, some access rules may give a participant full access to all room status values. In some implementations, the physician or recruiter may or may not be a virtual room participant depending on the status of the virtual room. Even if the physician or recruiter is not a virtual room participant, the physician or recruiter may still be a team participant, with access to the subject-centered chat, which is a communication channel.

Some access rules apply to a virtual room with a particular status. In other words, each room status for a particular enterprise may have one or more associated access rules. The access rules that have an associated room status may associate certain roles with the room, the participant-centered chat, or both, depending on the room status. The roles can be, e.g., a nurse, a physician, a specialist, interviewer, guide, etc. Thus, based on the room status of a particular virtual room and the access rules for that status, a particular user may become a room participant and/or a service team participant. Consequently, some enterprise personnel may be invited to/have access to a particular virtual visit object because of a combination of the room status, their role, and a schedule. For example, in a hospital setting a particular nurse may be assigned to a particular virtual room in a virtual visit object because the nurse is on-call at a particular time for a particular floor and the virtual room has a ‘recovery room’ status. Thus, a virtual visit object in combination with the access rules can enable people assigned to the virtual room to change automatically throughout the lifecycle of the virtual visit object. In this sense, the subject participant is the center of the virtual visit object and never changes, but the purpose (status) of the virtual visit object, and thus the participants in the service team and in the room, change around the subject.

The access rules may also define medical records and other information of the enterprise accessible to the virtual object. Such access rules may relate to the room status, to the subject participant, or relate to type of the virtual service (e.g., outpatient visit, rehabilitation visit, ICU visit, etc.). For example, access rules may enable a patient to access a daily menu offered by a hospital and select meals for the day for a virtual room with a room status of ‘recovery room’, ‘observation room,’ etc., or may enable a new student to select a break beverage As another example, the access rules may identify a form (i.e., data elements to be collected) to be completed by the patient or the job applicant for a virtual room with a ‘waiting room’ status. In some implementations, one or more of the access rules may be programmatic, e.g., integrated into program code for a user interface configured to support a particular room status.

The virtual visit object can also include a subject-centered chat. A subject-centered chat excludes the subject participant (e.g., the patient, the job interviewee, etc.) as a chat participant. Instead, a subject-centered chat includes communications (chats or messages) related to the subject participant among the team currently invited to the virtual visit object. The subject-centered chat messages are linked to the virtual visit object, which enables the messages to persist even after a participant no longer has access to the virtual visit object. As indicated above, the service team participants may change based on the room status of the virtual object. But because the subject-centered chat messages are linked to the virtual visit object, a new team participant is able to see what occurred prior to the participant joining the service team for the virtual visit object. This facilitates continuity of care, enabling care providers to get up to speed more quickly even in an environment where the providers are physically distant. Furthermore, because subject-centered chat messages are tied to the virtual visit object they have a limited life; e.g., they are atomically unavailable to the care providers after the virtual visit object expires. In some implementations, a virtual visit object is deleted after the virtual visit object expires. In some implementations, some elements of a virtual visit object may be added to an audit log before the virtual visit object is deleted.

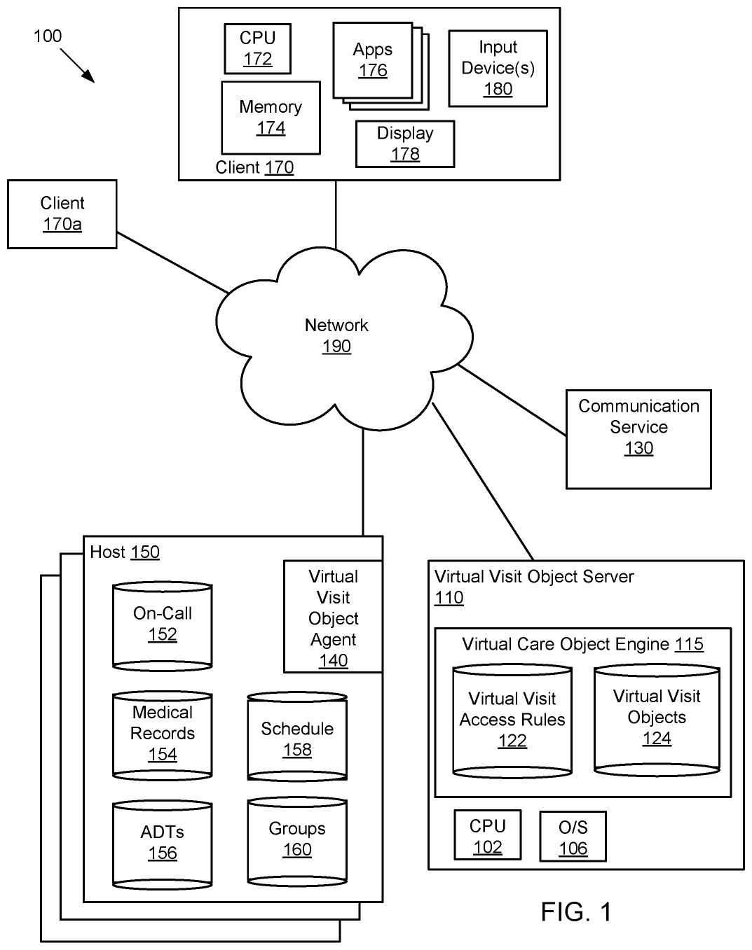

FIG. 1 describes a high-level depiction of a virtual visit object environment 100, according to a disclosed implementation. For ease of explanation, the environment 100 is described with respect to a medical setting, but implementations have applications beyond telemedicine visits and medical settings. For instance, the virtual visit object can be adapted to a human resources environment, e.g., virtually onboarding new employees, virtually interviewing job applicants, etc., to an academic environment, e.g., virtual conferences between parents and one or more education personnel, etc. Thus, reference to a service team can be generalized to any team, a medical provider generalized to any other role appropriate for the purpose of the visit (e.g., an HR manager, an interviewer, etc.), a patient to any subject participant, etc.

The virtual visit object environment 100 may include a number of computing devices that are in data communication with each other through a network 190 or a series of networks 190. The network 190 may include the Internet, a local area network (LAN), a wide area network (WAN), a cellular network (e.g., 5G cellular), an intranet, etc., or some combination of these. For example, client 170 may communicate with one or more remote computers, such as host 150, virtual visit object server 110, communication service 130, etc. via a LAN, a cellular network, an intranet, or the Internet. In some implementations, the network 190 represents more than one network, e.g., the Internet and a WAN or the Internet and a WI-FI and/or a cellular network.

The computing devices in environment 100 may include one or more hosts 150. The host 150 is a computer system operated by an entity providing services, e.g., an enterprise such as a hospital, physician's office, a human resources department, a rehabilitation provider, a training department, etc. The host 150 may represent a distributed computing system. The host 150 may represent a cloud-based system. The host 150 may be an example of an electronic medical records system (EMR), a personnel system, etc. The host 150 may require its users (e.g. employees or customers) to login with login credentials (typically a username and a password or token) before accessing the records and applications supported by the host 150. Thus, the enterprise host 150 may include one or more computing devices, such as a computer, a server, a rack of servers, a number of communicatively connected distributed servers, a mainframe, etc., that has one or more processors (e.g., a processor formed in a substrate) configured to execute instructions stored in one or more memories, such as main memory, RAM, flash, cache, or disk. The instructions may be stored in a runtime environment, functional modules and/or engines, e.g., applications, and may provide functionality to support the functioning and operation of a business entity, e.g., typical hospital applications, typical business applications, typical diagnosis, prescription, and billing applications of an EMR, etc. For ease of discussion in illustrating how a virtual visit object integrates with existing systems, e.g., with host 150, host 150 is described in the context of a medical provider, but implementations are not so limited. For example, the host 150 may include several data stores that support the applications running on the host 150. Examples of such data stores include on-call records 152, medical records 154, group records 160, schedule records 158, and ADT (Admission, Discharge, Transfer) records 156.

In some implementations, the host 150 may include a virtual visit object agent 140. The virtual visit object agent 140 may be configured to facilitate communication with a virtual visit object server 110. The virtual visit object agent 140 enables the virtual visit object server 110 to connect to data on host 150. For example, the virtual visit object agent 140 may include application programming interfaces (APIs) that enable the virtual visit object server 110 to send requests for data from the data stores at the host 150. In some implementations, one or more of the APIs may be offered by the enterprise, e.g., native to the host 150. In some implementations, one or more of the APIs may be installed at the host 150 as part of integrating the virtual visit object server 110 with the host 150. As another example, the virtual visit object agent 140 may be configured specifically for communications with the virtual visit object server 110. Put another way, in some implementations, the virtual visit object agent 140 may be configured to respond only to requests originating from the virtual visit object server 110. In some implementations, the virtual visit object agent 140 may facilitate communications with the virtual visit object server 110 across a firewall, or similar mechanism, protecting host 150.

The computing devices include the virtual visit object server 110. The virtual visit object server 110 may include one or more computing devices, such as a computer, a server, a rack of servers, a number of communicatively connected distributed servers, a mainframe, etc., that has one or more processors 102 (e.g., a processor formed in a substrate) configured to execute instructions stored in one or more memories, such as main memory, RAM, flash, cache, or disk. The instructions may be configured into applications, modules, or engines configured to perform operations. The virtual visit object server 110 may include an operating system 106. The virtual visit object server 110 may include virtual visit object engine 115. The virtual visit object engine 115 may be configured to implement virtual visit objects 124, to provide user interfaces for defining virtual visit access rules 122, to provide user interfaces for accessing virtual visit objects 124, and to communicate with other computing devices, such as host 150, communication service 130, and/or client 170.

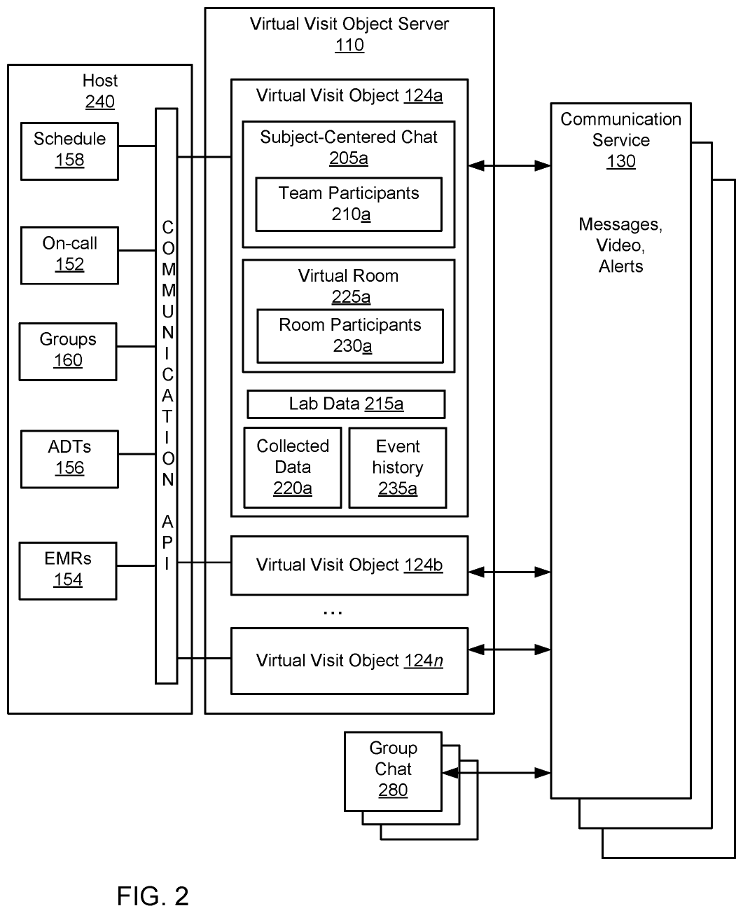

The virtual visit objects 124 represents a data store configured to store instances of virtual visit objects. As discussed in more detail with regard to FIG. 2, a virtual visit object may include a virtual room instance and may also include a subject-centered chat instance. As used herein, reference to a virtual room or virtual visit object for a subject participant is understood to refer to an instance of the virtual room or an instance of the virtual visit object. Thus, a virtual room for a subject and virtual room instance are understood to refer to a stored data item generated for the subject. The virtual room serves as the communication hub for the subject of the virtual visit object. The subject-centered chat service as a communication hub for team members about the subject of the virtual visit object. What communication is available to or takes place in a virtual room is dependent on the current purpose of the virtual room reflected by the room status. The room status thus reflects the function of the virtual room at the current time. The room status can determine what data is available in the virtual room. Some of the data may be available for update, e.g., a patient requested to complete or verify data. Some of the data may be available for viewing and/or for supporting actions within the virtual room. The room status may determine participants invited to the room. The room status may determine participants invited to a subject-centered chat. While described as a subject-centered chat, in a medical environment, the virtual visit object may be understood to have a patient-centered chat, or in a human resources environment the virtual visit object may be understood to have a candidate-centered chat, etc. One or more participants may not be based on the room status but may be based on association with the subject (e.g., patient, job candidate, student) of the virtual visit object. The virtual visit object engine 115 may apply virtual visit access rules 122 in view of the room status to determine participants, to configure user interfaces, to determine what data to access or collect etc.

The virtual visit access rules 122 represent configurable rules for determining access to, functionality of, and data for a virtual room instance. An enterprise, e.g., a medical institution (an EMR, hospital, EHR vendor, etc.), a medical provider, HR department, etc., may define rules for different types of virtual rooms, e.g., a waiting room, an exam room, a consultation room, an emergency room, a recovery room, an interview room, etc. The rules may define who has access to the room based on room status. Such rules may be referred to as room access rules. The virtual visit access rules 122 may define access to a room using room status and a role. A role represents the purpose of the service team member (e.g., nurse, social worker, physician, etc.) A user (team member) may be assigned to a role for a particular virtual visit object. For example, a particular physician may be assigned the role of primary care physician for a virtual visit object by association between the role and an identifier for the particular physician in the virtual visit object. A service team member may be assigned to a role based on the title for the role and a schedule. For example, a particular care member may be identified as the on-call floor nurse, or as a dietician assigned to the hospital floor, as a technician assigned to the room number, as a receptionist assigned to the front desk for the day, physical therapist from practice X, as an interviewer for a first time slot, etc. Thus, the virtual visit object engine 115 may obtain data from host 150 (e.g., on-call records 152, schedule records 158, etc.) or from other data stores (not illustrated) to determine which users are considered room participants and/or team participants. Some virtual visit access rules 122 may also allow creating additional roles for a particular virtual visit object, such as, for example, patient requested participants (e.g., family members). Thus, for example, depending on the room status, the access rules may enable a patient to invite additional users to the virtual room. Some access rules may enable a physician (or other participant) to invite other service team participants, referral invitees, health insurance invitees, pharmacy invitees etc., to either the virtual room or the subject-centered chat. Whether or not participants may invite other participants may be determined by room status (e.g., the access rules may enable or disable such invitations based on room status and/or role).

Some virtual visit access rules 122 may, for some roles, define data access rights. Such rules may be referred to as room participant access rules. Data access rights, also referred to as level of access, include the scope of accessible data content, time period of enabled access, privacy and audit settings for the data access, etc. In one implementation, the patient (or other subject participant of the virtual visit object) may be enabled to configure the data access rights of participants invited by the patient (e.g., enable unmuting of a microphone). In some implementations the visit access rules 122 may enable a room participant, such as the subject participant, to determine whether certain events are recorded in the event history. For example, a patient may have the ability to save video and/or audio from teleconference or video conference events, where the default is that such events are not saved in the event history. Some virtual visit access rules 122 may define, for the room status, what information might be available to the virtual visit object. Some data may be collected data. For example, a patient may be asked to fill out insurance forms or a medical history in a virtual room with a ‘waiting room’ status. The virtual visit access rules 122 may identify a form, user interface, etc., used to collect the data. Any data that is collected in a virtual room is stored as part of the virtual visit object. In some implementations, some of the collected data may be pushed to host 150, e.g., for storage in medical records 154. In some implementations, the room participant access rules may enable a subject participant and/or a team member (e.g., primary physician) to control what data items an invited room participant may access. One or more of the room participant access rules may be programmatic, or in other words implemented in code used to select and generate a user interface. For example, each room status may be associated with code configured to provide a user interface that enables room participants to interact. The user interface presented for a room status may be dependent on the participant's role and the room status, and in this sense is a room participant access rule. Furthermore, some data elements may be included in the user interface by application of one or more configurable room participant access rules. For example, a system administrator may be able to select data elements for collection in the user interface, data elements to present in the user interface and may be able to indicate which roles have access to which elements.

The computing devices in the virtual visit object environment 100 also includes client devices, such as client 170 and 170a. Client 170 provides a user interface, for example via a browser, through a mobile application, etc., for a human user to access various capabilities supported in the virtual visit object environment 100. The client 170 may be any personal computing device, e.g., laptop, tablet, smart phone, cellular phone, smart watch, smart glasses, television with a processor, etc., The client 170 may be a computing device that is capable of receiving messages, such as text messages, short message service (SMS) messages, secure message service, instant messages, email, etc. In some implementations, the client 170 may be a mobile device identified by a phone number or user login. The client 170 may include a central processing unit 172, which may be one or more processors formed in a substrate configured to execute one or more machine executable instructions or pieces of software, firmware, or a combination thereof. The processors can be semiconductor-based—that is, the processors can include semiconductor material that can perform digital logic. The client 170 can also include an operating system and one or more computer memories 174, for example a main memory, configured to store one or more pieces of data, either temporarily, permanently, semi-permanently, or a combination thereof. The memory 174 may include any type of storage device that stores information in a format that can be read and/or executed by the central processing unit 172.

The client 170 may also include one or more applications 176. The applications 176 may be mobile applications, e.g., applications downloaded from an app store that perform a specific function. Thus, for example a client 170 may include an application 176 that works with the virtual visit object engine 115, communication service 130, and/or host 150 to present one or more user interfaces on the client 170. The applications 176 may include a browser-based application, which presents user interfaces (e.g., from virtual visit object engine 115) using a browser. Thus, the applications 176 may also include an Internet browser. The client 170 may also include a display 178, such as an LCD or LED display, a touch screen display, etc., that displays images and text rendered by the applications 176. The client 170 may also include one or more input devices 180, which can include a touch-sensitive display, a mouse, a keyboard (including a keyboard displayed on display 178), etc. A client (e.g., client 170, client 170a, etc.) may be used by any participant of a virtual visit object. The virtual visit object engine 115 may initiate display of user interfaces on display 178, e.g., via instructions provided to an application 176. Client 170 may communicate with virtual visit object server 110, communication service 130, and/or host 150 over network 190.

Although illustrated as a separate server in FIG. 1, in some implementations, one or more elements of virtual visit object server 110 may be combined with host 150. In some implementations, one or more elements of virtual visit object server 110 may be combined with communication service 130. In some implementations, one or more elements of communication service 130 and one or more elements of virtual visit object server 110 may be combined with host 150. Thus, implementations are not limited to the exact configuration of the virtual visit object environment 100 illustrated.

FIG. 2 illustrates a depiction of additional elements in a virtual visit object environment 100, according to a disclosed implementation. In FIG. 2 some of the components illustrated in FIG. 1 are represented in additional detail and others are omitted. The environment depicted in FIG. 2 illustrates integration between the virtual visit object server 110 and host 240 and also with communication service 130. The host 240 may represent an existing electronic medical records system and can be one example of host 150. Integration of virtual visit object server 110 with host 240 provides virtual visit objects 124 (e.g., virtual visit object 124a, virtual visit object 124b, virtual visit object 124n, etc.) for subject participants serviced by host 240. As illustrated in FIG. 2, the virtual visit object server 110 can include several virtual visit objects 124. For ease of the discussion, where reference is made to virtual visit object 124a, virtual visit objects 124b to 124n are understood to have similar structure and offer similar functionality, but for their own respective subject patients and service teams.

The virtual visit object server 110 provides patient and medical personnel dashboards based on the virtual visit object 124a and the room status of the virtual room 225a and facilitates communication between participants for the virtual visit object, e.g., chats, teleconferences, and video conferences. The virtual visit object engine uses communication APIs to obtain data from and/or provide data to the host 240. The virtual visit object engine 115 may also use a communication service 130 to enable participants associated with a virtual visit object 124a to send messages, images, documents, etc., to one or more participants in the virtual visit object 124a (e.g. room participants 230a) or to send messages, images, documents, etc., to one or more team participants 210a. Team participants 210a may initiate a video conference or teleconference with room participants 230a within the virtual room 225a.

Virtual visit object 124a may include different kinds of communication channels (chats), e.g., a general chat that room participants 230a can view and participate in and a team chat between non-subject participants (team participants 210a) for the virtual visit object 124a. The communications in these chats can be recorded in event history 235a. In one implementation, details of video or teleconference sessions may be included in the general chat. The details include who participated in the video session, when the session started (i.e., a timestamp for the start of the video call), and how long the session lasted (e.g., as calculated from a timestamp for the end of the call or as the duration), etc. In some implementations, the audio and video of a video call or teleconference may not be recorded as part of the virtual visit object 124a. In some implementations, the audio and video may be included, e.g., in event history 235a. In some implementations, the general chat data is stored in event history 235a. In some implementations, when the virtual room 225a changes its room status, general chat data is moved to the event history 235a. In some implementations, general chat data is linked to the room status. Linking the general chat data can enable the virtual visit object engine 115 to display chat data relevant to the room status, or by room status, or to enable searching of general chat data by room status in the event history 235a. The room status may provide context for the chat. In some implementations, general chat data is not stored in event history 235a for some room statuses. In some implementations, only certain roles have access to general chat data associated with past room statuses. In some implementations, only certain roles may have access to event history 235a. Access may be governed by virtual visit access rules 122.

The subject-centered chat 205a may persist while the virtual visit object 124a is active. The subject-centered chat 205a may be accessible to the team participants 210a, which exclude the subject participant (patient) and subject invitees. This enables care providers to leave notes to facilitate shift changes and to consult with one another and helps bring a new service team member up to speed. The team participants 210a are determined based on the room status and the applicable virtual visit access rules 122. Thus, individuals do not need to be explicitly invited to the subject-centered chat 205a. This differs from a group chat, e.g., group chat 280, where individual participants must be expressly invited to join and remain until expressly removed. The role-based inclusion in the team participants 210a means the service team is data-driven with automatic handoff. This facilitates communication and improves care, especially in an emergency or highly fluid situation.

In one implementation, the virtual visit object 124a is stored separately from electronic health records and is only available for a short time, e.g., while the virtual visit object is active or for a period of time after all the service sessions end. Thus, in some implementations, the virtual visit object 124a is deleted, e.g., upon closing the virtual visit object or after the period of time after the virtual visit ends. In another implementation, the virtual visit object is preserved in a virtual visit object record (not shown), which can include a partial or a complete data record of the virtual visit object 124a for future reference, for asynchronous collaboration with other service teams or providers, to facilitate care payments, etc. In some implementations, the virtual visit object 124a, or portions of a virtual visit object 124a, may be written to an audit record.

In some implementations, the virtual visit object 124a may include collected data 220a. The collected data 220a can include data obtained from an enterprise system, such as host 240, and data collected in the room, for example using a form or via chat. Collected data 220a represents files (images, documents, PDFs) collected in the virtual room, e.g., as attachments for chats occurring in the virtual room, as form data elements provided in a virtual room, etc. The virtual visit object 124a may thus include data from subject-centered chats (205a) and/or chats with the patient that occurred within the virtual room (225a). The virtual visit object 124a may include team participants, i.e., team participants 210a, and room participants, i.e., room participants 230a. Team participants 210a can utilize subject-centered chats 205(a). Some team participants 210a may be invited based on a status of the virtual room 225a and enterprise rules, e.g., access rules 122. Some team participants 210a may be invited based on the type of the virtual service or the relationship to the patient (e.g., primary care physician, surgeon performing a surgery) regardless of room status. A team participant may leave the team participants, but if all team participants leave the virtual visit object may close. The team participants are also room participants. Depending on the status of the room, the virtual patient room may enable room participants to communicate with each other, e.g., via a chat in the room or via a teleconference in the room. Team participants may chat with each other via subject-centered chat. Such chats are not part of the virtual room.

The virtual visit object may also include lab data 215a and other medical data related to the patient. The lab data 215a can be stored as part of the virtual visit object 124a or the virtual visit object 124a may access the results from a separate electronic medical records system. For example, an existing medical records system may provide an API that allows a virtual visit object to access certain data. Thus, a virtual visit object 124 encapsulates and organizes data related to a virtual visit in a way that provides automatic access to the data to those who need it, as well as to patient-invited invited participants (depending on the room status). In some implementations, other data may be associated with the virtual visit object 124a.

The virtual visit object 124a includes an event history 235a. The event history 235a provides a complete picture of each service session of the virtual visit, including participant changes, the record of each service session that are completed throughout the visit. For instance, details may include when the virtual visit object was created, when a teleconference or videoconference was initiated, when a change in room status occurred, who initiated a video conference or changed the room status, which participants (users) were in the room and/or participated in communications in the room, when communications occurred, what role a particular participant fulfilled, etc. Conventionally, in a hospital setting such a record does not exist, as the data and participants in the virtual visit get filtered to different systems or are not collected at all. The event history (e.g., 235a) organizes information around the patient, not around different hospital departments, specialists, etc. Moreover, because the virtual visit object 124a is ephemeral, the event history 235a can capture information that may not be important enough to a medical provider to capture, but which improves patient care during the episode of medical care. In some implementations, the communications that happen in the subject-centered chat 205a and in the virtual room 225a may not be included in the event history 235a. In some implementations, metadata about some of the communications (e.g., participants, time, type of communication) may be captured in the event history 235a. Some or all of these details may be pushed to host 240 for inclusion in electronic medical records, in accordance with relevant rules, procedures, and regulations. In some implementations, the virtual visit object 124a may encapsulate details about the virtual visit and/or a service session, such as, for example, enable access to a recorded video conference of the service session, or other sensitive data, such recordings or data may not be permanently preserved for security or privacy reasons.

In some implementations, whether a chat is available as part of the virtual room 225a may depend on the room status. In some implementations, participants in the chat may depend on the room status. For example, in some implementations a virtual room 225a with a ‘waiting room’ status may not have chat capability, or may have chat capability but only with a receptionist. Similarly, a virtual room 225a with a ‘recovery room’ status may not have chat capability for the patient. However, even when a virtual room 225a lacks a chat capability, the virtual visit object 124a may support the subject-centered chat 205a.

Implementations enable a patient to request a status change of the virtual room 225a. For example, in some implementations a patient may not be able to contact (chat with) certain service team members within the virtual room 225a, but may request that the service team member change the status of the room. For instance, the patient may request the status of the virtual room 225a be changed to an exam room, which may initiate a teleconference with a service team member. Depending on the room status and access rules 122 applicable to the virtual visit object 124a, the patient may be enabled to text some or all of the caregivers associated with the virtual visit object 124a and/or participate in a teleconference with some or all of the caregivers. During a teleconference each participant may have access to the data associated with the virtual visit object 124a, e.g., the event history 235a, which may include data collected within the virtual visit object 124a as well as data indicated as relevant to the room status. For example, the applicable rules 122 may indicate that more data from the electronic medical records 154 is available in a virtual room 225a when the status is ‘consultation room’ than when the room status is ‘exam room’ or ‘recovery room.’

A patient may request that a status of the virtual room be changed. For example, after a telemedicine visit the physician may ask the patient to call if things get worse and the virtual room 225a may have a room status of ‘post-consultation’ or something similar. The virtual visit object 124a may have this status until a predetermined time after the telemedicine visit. If things do get worse, the patient may request a follow-up virtual exam. This request, once accepted by the physician, may correspond to a request to change the status of the virtual room to ‘exam room’ or something similar. Such a request may be prioritized over initial telemedicine visits, e.g., in a list of virtual rooms for a provider. Further, the existing virtual visit object 124a includes the event history 235a, so the physician has access to the record of the prior telemedicine visit in accepting the room status change. Thus, the disclosed infrastructure supports a process that makes the continuity of care more seamless, providing the support for a workflow that did not previously exist and makes efficient use of resources.

The dynamic nature of a virtual room of a virtual visit object 124 is demonstrated in another example, where a patient arriving at the emergency room may have a virtual visit object created. The initial status of the virtual room may be ‘waiting room,’ which may provide the patient access to a receptionist via chat while in the waiting room, and may be associated with one or more forms for the patient to fill out in accordance with applicable virtual visit access rules. Some of the information available to the patient in this virtual room can include an estimated wait time, if one exists, for example. Once the patient has been admitted, the room status of the virtual visit object may be changed to ‘er evaluation’, which may invite an emergency room nurse and an attending physician in accordance with the virtual visit object access rules for the hospital. The attending physician may be remote from the ER, e.g., participating in the evaluation via a video call that occurs in the virtual room. As part of the evaluation, the attending physician may determine an x-ray is needed. This may change the room status to ‘x-ray,’ which may uninvite the emergency room nurse as a team participant, but invite an x-ray team member (e.g., x-ray technician) as a team participant. The x-ray technician may be able to see any notes the nurse or physician put in the subject-centered chat and may be able to ask the attending physician for any needed clarification via the subject-centered chat.

If the patient is then admitted, the room status of the patient's virtual visit object may be changed to ‘observation’ or ‘recovery’ or some other status. This may remove the x-ray technician from the team participants (or in other words revoke the technician's access) but invite a floor nurse and an attending physician (who may be the same as or different than the attending physician in the ER) in accordance with the applicable virtual visit object access rules. The actual x-ray technician, nurse, and physicians who have access to the patient's virtual visit object, and thus the virtual room, may be based on a schedule, such as a nurse assigned to the room or floor via a schedule at the hospital. The primary care physician of the patient may be invited to the virtual visit object as a team participant regardless of room status. Any service team participant may have access to the event history, facilitating care and reducing errors. The patient may invite family members to the virtual room regardless of room status. Applicable virtual visit object access rules may determine what data and what actions are available to the patient and/or to the participants the patient invites to the virtual room. Thus, for example, when an attending physician comes in to talk to the patient, the physician may initiate a teleconference in the virtual room so that the patient's participants can participate even if they cannot be present at the hospital.

The virtual visit object generated for the patient may also include or may fetch lab results or other medical records for the patient. The virtual visit object may also include data entered by or provided by the patient (e.g., data from intake forms, data from special forms, documents relate to the patient medical history, electronic health records etc.).

The virtual visit object, e.g., 124a, may exist for the duration of a virtual visit. In some implementations, once the virtual visit object is closed (e.g., at the end of the last service session) the virtual visit object may be deleted. In some implementations, the deletion may occur after some predetermined time, e.g., according to the rules 122 associated with the type of the virtual visit.

In some implementations, the virtual visit object may be exported to a data capsule which includes part or all the data elements presented or generated in the virtual visit object. In some implementations, the data capsule is operable to re-create a read-only care record object, such as, for example, as a standard record keeping for the medical providers. In another implementation, part or all contents of the data capsule may be selectively shared by the medical provider.

The virtual visit object engine 115 may provide access to the virtual visit objects 124 via various user interfaces. In some implementations, the user interfaces may be browser based. In some implementations, the user interfaces may be via a mobile application installed on a client. In some implementations, access to the virtual visit object may occur after authentication, e.g., via a credential login (e.g., username and password/biometric factor, etc.) In some implementations, a patient or patient invitee may access the virtual visit object via ticket access, so that a login/account creation is not required for the patient to access the virtual room. In such a system, any personal data may be anonymized. In such an implementation, the virtual room user interface for the subject (e.g., patient) may be configured to present protected information and include protected identifiers when accessed via authentication. In some implementations, the ticket-based access may be generated as part of an invitation, the invitation being sent to an address associated with the patient. When presented with a request for information that includes a ticket, some implementations may gather the information to be included in the patient's view of the virtual room, but may generate mock identifiers for each protected identifier to be provided as part of the patient's view of the virtual room. A mock identifier is a temporary system-generated identifier used to replace an authentic identifier. Mock identifiers are only valid for a specific session. In some implementations, the system may generate an entry in an identifier map for a session identifier, which is extracted from the request for information. The entry may include the mock identifiers generated and pair each mock identifier with the protected identifier it replaces. Thus, the entry may contain one or more mock identifier-protected identifier pairs. In addition, protected information may be removed from the information provided in the patient's view of the virtual room. The protected information may be provided if a user logs in via authentication but removed if a user accesses the user interface via ticket presentation. Protected information includes any personal health information, any personally identifiable information, etc. Protected identifiers are protected information that is included in the information used by the virtual room when accessed via ticket presentation, but such identifiers are mapped to mock identifiers tied to the session. Thus, the protected information is anonymized by either removal or replacement. Implementations can also offer access to a virtual patent room via authentication, such as logging in to a user account.

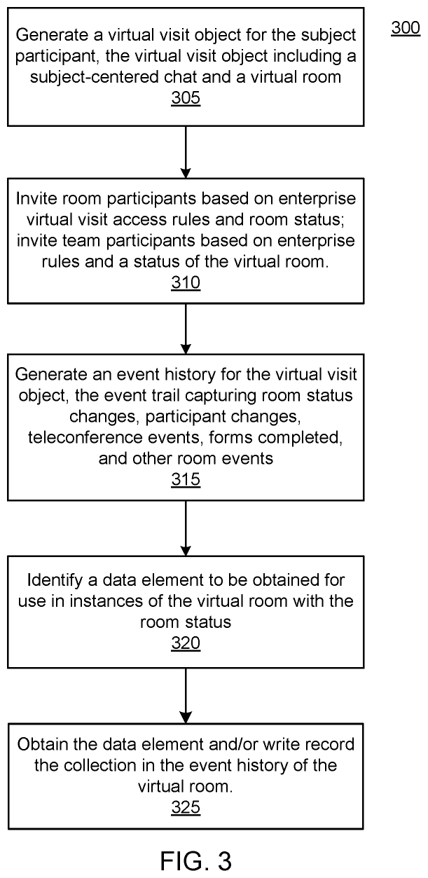

FIG. 3 illustrates a flowchart of an example process 300 for creating a virtual visit object, according to a disclosed implementation. Process 300 may be performed by a system executing a virtual visit object engine, such as environment 100 of FIG. 1. Process 300 creates a persistent data container relating to a virtual visit.

Process 300 begins with creating a virtual visit object for a subject participant's virtual visit (305). The virtual visit object includes a subject-centered communication channel with team participants and a virtual room with room participants, the virtual room including its own communication channel. The room participants include the team participants but also include the subject participant and anyone the subject participant invites to the virtual room. The team participants may be dependent on a status of the virtual room. Each virtual room status may have rules (access rules) that apply to instances of virtual rooms with that status. The rules may be set by enterprises (e.g., care providers) and may relate to what data is available from the enterprise's electronic records (e.g., via APIs), what data is to be collected in the room (e.g., an intake form, an insurance form, etc.), what participants are automatically invited to the room as team participants, etc. Once a virtual visit object is generated, the system may invite room participants. (310). In some implementations, the participants may be invited via a communications identifier, e.g., an email or a mobile phone number.

In some implementations, the system may invite participants via a role. For example, the system may include rules that identify one or more roles associated with the status of the virtual room. The system may match up a role for the virtual room status with a currently scheduled employee with that role (e.g., the on-call nurse). In some implementations, a team member may be invited based on the type of virtual service (e.g., doctor office visit, scheduled surgery, etc.) The system may generate an event history for the virtual visit object (315). The event history captures information about who is/was a team participant or a room participant, what data was collected, what communications occurred, virtual room status changes, as well as other events related to the virtual service. The event history enables new room or team participants to quickly assess the current state of a room and/or the subject participant and may be used to trigger other events, e.g., the start of a teleconference etc.

In some implementations, the system may identify a data element, e.g., a medical data element, an orientation data element, etc., to be collected in stances of the virtual room type. (320). For example, some virtual room types may have an associated form (or forms) with one or more data elements to be collected. The subject participant may be asked to complete the form before being “seen” in the virtual room. The data elements collected may be stored as part of the virtual visit object (325). In some implementations, the data elements collected may be stored in the event history associated with the virtual visit object. In some implementations, some or all of the data elements collected as part of the virtual visit object may be transmitted/exported to an external system, e.g., a physician's electronic medical records (EMR) system. In some implementations, the virtual visit object may also pull data elements for the subject participant from an external electronic records system (320). Such data elements can be displayed as part of an interface for the virtual room.

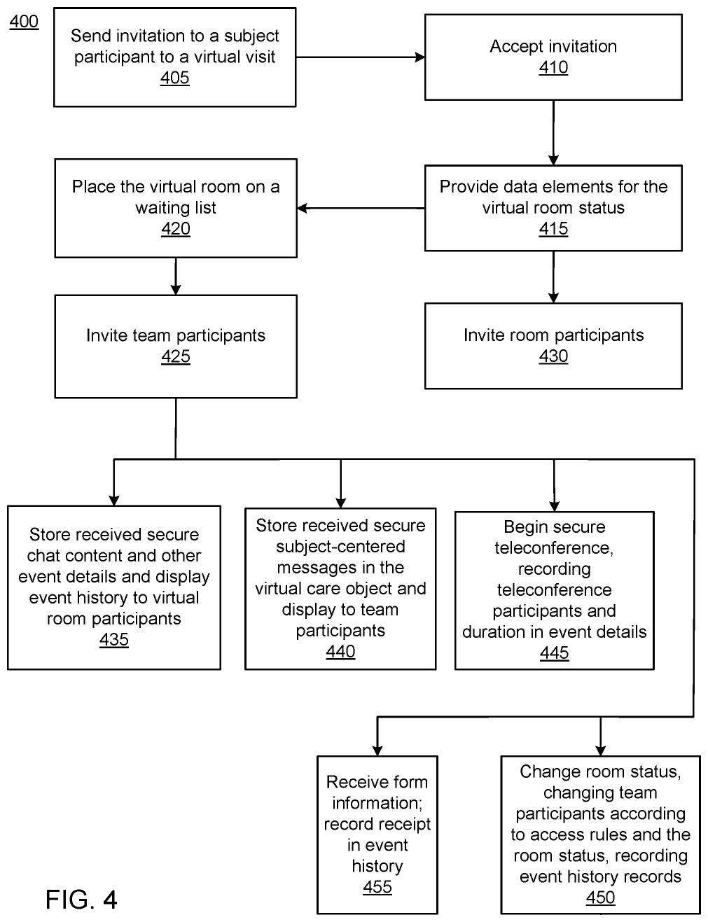

FIG. 4 illustrates flowchart of an example process 400 for providing a virtual room, according to a disclosed implementation. Process 400 may also be performed by a virtual visit object engine. Process 400 begins by opening a virtual visit object and sending an invitation to the virtual room to the subject participant (405). Once the virtual room is open, and depending on the virtual room status, the system may add the virtual room to a waiting list, e.g., for a physician, for a recruiter, for a guide, for a teacher, etc. (420). A subject participant may accept the invitation (410). In some implementations, the system does not add the virtual room to a waiting list until the subject participant has accepted the invitation. In some implementations, the virtual room may have a status that is associated with a form (e.g., a ‘waiting room,’ welcome room,' or ‘intake room’ status). The subject participant may be asked to fill out the form (415), providing data elements associated with, or in other words specified as to be obtained in, a virtual room with the specified status. The subject participant may also invite other participants (room participants) to the virtual room (430), e.g., a family member, guardian, a friend etc.

The system or a team participant may also invite other team participants (425). In some implementations, the virtual room status may be associated with rules that specify participants by role, e.g., nurse on call, receptionist, guide, etc. Thus, some implementations may have access to schedules and contact information stored in the enterprise's system, e.g., via API calls. In some implementations, room access rules may prohibit invitation of additional room participants. This may depend on room status. In some implementations, team access rules may prohibit invitation of additional team participants. This may also depend on room status. Several activities may occur after a virtual room has been opened, depending on the room status. For example, the subject participant and any other room invites may send and view chat messages (including images) in the room (435). Such chat messages may be tied to the virtual room and visible and accessible to room participants until the room is closed and/or the room status changes. In another example, team participants may have a separate subject-centered chat room that includes team participants (e.g., medical care team, interview team) but excludes the subject participant (e.g., patient, job candidate) and any non-team participants (such as the patient's invitees) (440). Such chat messages may be associated with the virtual visit object and be available to any team participants until the room closes and/or the room status changes. In some implementations, a video or tele-conference may be initiated (445). The teleconference may include secure video feeds for all participants currently in the room that accept the video conference. The system does not record the video or audio of the conference but may record the participants and duration of the call, e.g., in an event history. The system may record the participants and duration of any video or teleconference that occurs in the room. In some implementations, the access rules may determine, based on room status, whether a teleconference or video conference is offered as an option within the room.

The virtual room may experience a change in status, which may result in a change in the team invites (450). Such room status changes may be recorded in an event history (event history records). The team participants for a particular room status may depend on application of enterprise rules (e.g., rules 122), the type of virtual visit, and the subject participant. The subject participant may provide information, e.g., menu selections, request for room status changes, etc., which may be recorded in the event history (455).

In some implementations, the virtual visit object (and the virtual room associated with it) may close after a designated participant (e.g., the medical provider or recruiter that opened the virtual room) exits the virtual room. In some implementations, the virtual visit object may close after all team participants have left. In some implementations, the virtual room instance may be closed automatically by an event, such as a discharge from the hospital, or change of the room status to a designated terminal room. In some implementations, the event that closes the room may be dependent on the type of the virtual service. For example, a virtual visit object for a primary health care visit may close when the physician leaves the team participants, while a virtual visit object corresponding with a hospital visit may remain open for the duration of the hospital visit. The virtual visit object may persist for some period of time after the virtual room closes. After the period of time the virtual visit object may be deleted. As long as the virtual room remains open, any of steps 425 to 455 may be available, depending on access rules and/or room status.

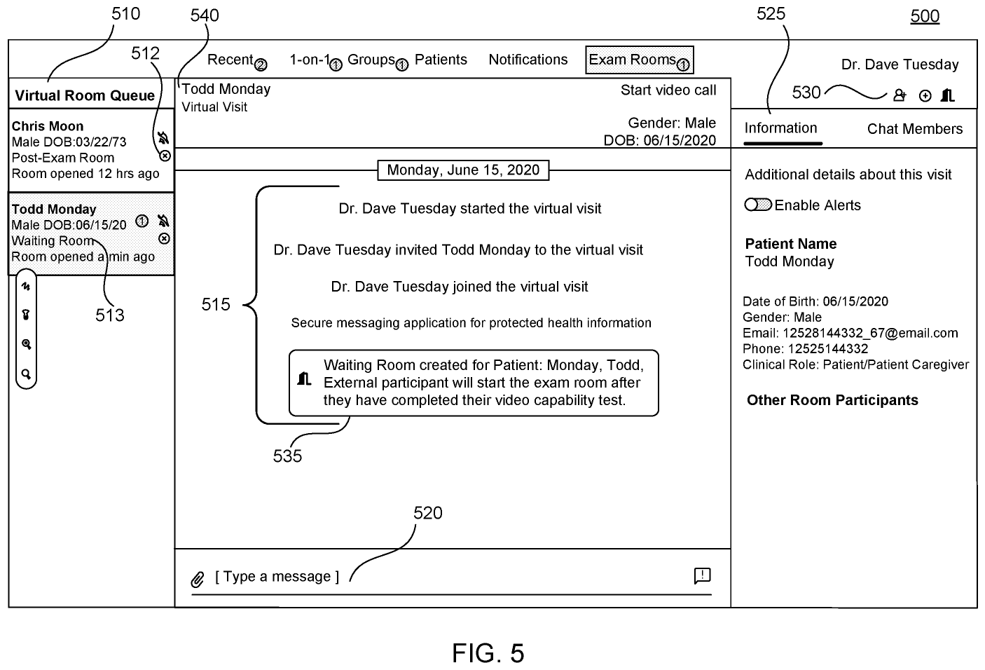

FIG. 5 illustrates an example user interface 500 for a physician initiating a virtual visit, according to a disclosed implementation. In the example of FIG. 5, a physician (Dr. Tuesday) has opened a virtual visit object for a patient, Todd Monday. The example user interface 500 is configured for a desktop device (e.g., laptop computer, desktop computer, etc.), but implementations may include an interface with similar data elements displayed differently for a mobile environment.

The example user interface 500 includes a main viewing area 540. The main viewing area 540 includes an event history 515 area. The event history 515 area may list some or all of the events recorded in the event history for the visit, e.g., in event history 235a. In the example of FIG. 5, the event history 515 area lists details about the start of the virtual visit and when the subject participant was invited. The example user interface 500 also includes a virtual room queue 510. The virtual room queue 510 may list some details about different virtual visit objects that the user (in this case Dr. Tuesday) is a participant in. In other words, the user interface 500 for a physician or other service team providers may include an interface element that enables a user to see and to navigate to different virtual visit objects.

As illustrated in FIG. 5, the virtual room queue 510 may include a summary of each active virtual visit object. The summary may include a subject participant identifier (e.g., a name of the subject or some other identifier of the subject). The summary may include a room status indicator 513. The room status indicator 513 may be text describing the current room status or may be a visual cue (e.g., a color, an icon) representative of the current room status. The summary may include a control 512 for ending the virtual visit. In some implementations, the virtual visit object may be terminated after some predetermined time, e.g., one day after being opened, one day after a last communication, one week after discharge, etc. The control 512 may be used by a participant to terminate the virtual visit object early. In some implementations, only certain participants may be allowed to close a virtual visit object early, e.g., a primary participant (such as the primary care physician, who may be a room participant regardless of status), an office manager, etc., and/or the subject participant. Thus, for example, selection of the control 512 in the example of FIG. 5 would close the virtual visit object for subject participant Chris Moon.

In some implementations, the example user interface 500 may enable a physician participant to change the virtual visit displayed in the main viewing area 540. For example, the summary may include a selectable area and selection of the selectable area may change the virtual visit object displayed in the main viewing area 540. Thus, if the physician selects “Chris Moon” the main viewing area 540 may display the event history for the virtual visit object for patient Chris Moon and messages sent via the messaging area 520 will be associated with the virtual visit object for Chris Moon.

The main viewing area 540 of the example user interface 500 may include a messaging area 520. The messaging area may enable room participants to send text communications, files, images, etc. within the virtual room. The messages (including attachments) may be transmitted using known or later developed secure messaging techniques. However, in some implementations, the messages are associated with the virtual room, not with a user account. In such implementations, the messages are not available after the virtual visit object ends, unless the virtual visit object is available via audit logs or the information has been pushed to a records system, e.g., an EMR.

The example user interface 500 also includes a virtual visit data panel 525. The virtual visit data panel 525 displays different data elements related to the virtual visit object displayed in the main viewing area 540. In the example of FIG. 5, the virtual visit data panel 525 displays subject participant information. This panel or other panels may also display data collected during the virtual visit (e.g., collected data 220a), lab results or other test results associated with the virtual visit, a resume, information from a resume, or a link to a resume associated with the virtual visit, etc. The virtual visit data panel 525 may list current room participants and/or team participants. In some implementations, the event history 515 may be a virtual visit data panel 525. The example user interface 500 may also include a new participant control 530. The new participant control 530 may enable one or more of the room participants to invite another person to the virtual room. The new participant control 530 may be available to some participants and not others. The new participant control 530 may be available for one room status but not another room status, e.g., depending on implementation and/or access rules.

The data and controls displayed as part of the user interface 500 may depend on the room status and the participant role. For example, because the virtual visit object displayed in the main viewing area 540 has a ‘waiting room status’ and the user has a physician role, the event history 515 may include one or more status messages 535. The status messages 535 may provide additional explanation for an event. In the example of status message 535, the waiting room creation event includes additional explanation that provides the physician with an indication of how things are progressing in the waiting room for the subject participant (the patient). In a telehealth environment, this can help the physician know who is ready to enter an exam room. In some implementations, a receptionist may be a room participant while the virtual room has a room status of ‘waiting room.’ The receptionist may receive messages (notifications) for messages sent by the subject participant while the virtual room has a ‘waiting room’ status. Thus, a user other than the physician can help the patient with intake forms, etc. before the physician is invited to the virtual room. In addition, a user with a receptionist role (rather than the physician) may instantiate virtual objects and invite the subject participants. Such an implementation may reduce the number of virtual objects in the physician's virtual room queue 515, so that only patients ready to be seen in an exam room are visible.

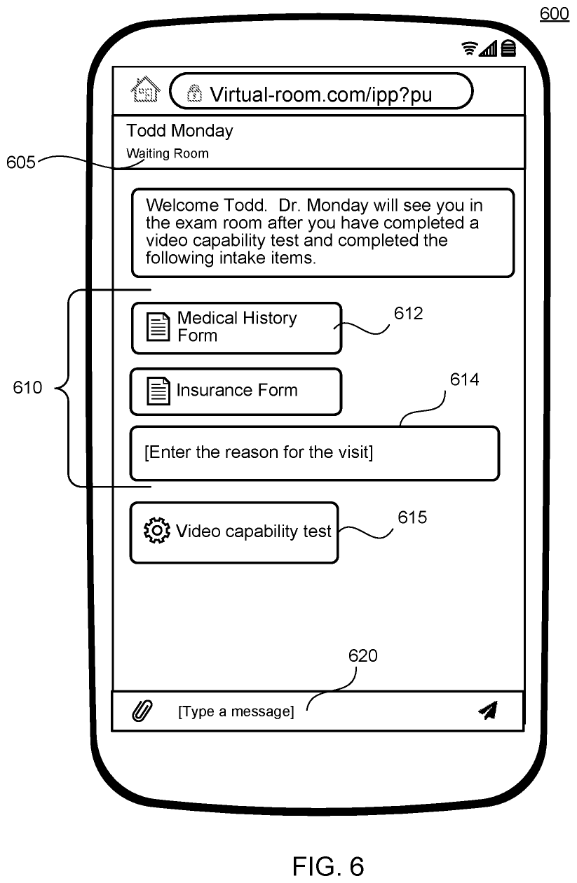

FIG. 6 illustrates an example user interface 600 for a patient in the virtual room with a ‘waiting room’ status, according to a disclosed implementation. In the example of FIG. 6, the subject participant (Todd Monday) has accepted an invitation to the virtual visit object and has joined the waiting room. In some implementations, as part of accepting the invitation the user may be required to authenticate to a user account. In some implementations, as part of accepting the invitation the user may use ticket-based authentication, which does not require a user account.

The example user interface 600 may be provided based on the room status (of ‘waiting room’) and the participant role (e.g., subject participant). Thus, the example user interface 600 represents a virtual room interface configured for room status (e.g., waiting room) and participant role (e.g., subject participant) while the example user interface 500 of FIG. 5 represents the virtual room interface configured for room status (e.g., waiting room) and participant role (e.g., physician). The example user interface 600 is configured for a mobile device (e.g., tablet, smartphone, smartwatch), but implementations may include an interface with similar data elements displayed differently for a desktop environment. Such an interface may include additional elements not illustrated in example user interface 600.

The example user interface 600 may include an indication of the room status 605. The indication of the room status 605 can be text based or a visual cue (color, icon, etc.), or a combination of these. The example user interface 600 may also include data items to be collected 610. These data items may include individual data items, such as data element 614, which represents a text box where the patient can enter a reason for the virtual visit. The data items may also include a plurality of data elements, e.g., collected via a form, such as data elements 612. In some implementations, the example user interface 600 may be configured to provide a pop-up window or another user interface where the plurality of data elements 612 are collected. In some implementations, the data items to be collected 610 may be based on room participant access rules applicable to the room status. In some implementations, the data items to be collected 610 may be based on a combination of room participant access rules applicable to the room status and the role of the participant. In some implementations, the data items to be collected 610 may be based on a combination of the room status and a data element related to the subject participant (e.g., a diagnosis for the patient, etc.). In some implementations, the patient must submit the requested data elements before moving the virtual room to a next room status.

The example user interface 600 may include a messaging area 620. The messaging area 620 may enable the participant to send text communications, files, images, etc. within the virtual room. The messages (including attachments) may be transmitted using known or later developed secure messaging techniques. However, in some implementations, the messages are associated with the virtual visit object, not with a user account. The messages may therefore be viewed by room participants. In some implementations, the system may associate messages with the room status. Thus, for example, messages sent in the ‘waiting room’ may be viewable while the virtual room has a room status of ‘waiting room’ but may not be visible once the room status changes. As another example, messages sent in the ‘waiting room’ may be viewable while the virtual room has a room status of ‘waiting room’ but may only be visible in an event history data panel or event history interface once the room status changes. The event history panel or event history interface may be configured to display messages by room status, regardless of the current room status. In some implementations, messages associated with the virtual visit object are not available after the virtual visit object expires.

The example user interface 600 may also include a video capability test control 615. The video capability test control 615 may determine whether the user device has software and hardware needed to participate in a video conference. In other words, the video capability test determines the device being used meets minimum video requirements. In some implementations, the user must complete this test (e.g., initiated by selection of the control 615) before the room status changes from ‘waiting room’ to ‘exam room.’

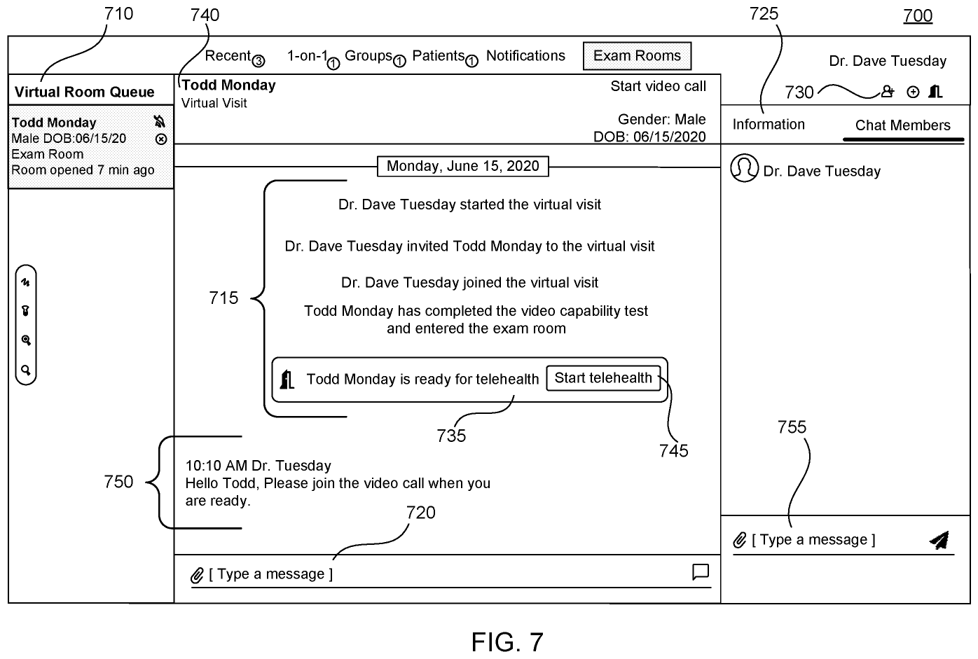

FIG. 7 illustrates an example user interface 700 for a physician in a virtual room with an ‘exam room’ status, according to a disclosed implementation. In the example of FIG. 7, the subject participant (Todd Monday) has successfully completed the video capability test control, which causes the room status of the virtual room to change from ‘waiting room’ to ‘exam room’. With this change in status, the system may automatically change the room participants. For example, if a receptionist initiated the virtual visit object for Todd Monday, as a result of the room status change the receptionist would be removed as room participant and Dr. Tuesday would be invited as a room participant. The invitation may result in the virtual visit object being added to a virtual room queue 710 for Dr. Tuesday. Room participants may also be invited based on access rules (e.g., room access rules) applicable to the room status. For example, Dr. Tuesday may be invited because Dr Tuesday is Todd Monday's primary care physician and the access rules indicate the primary care physician should be invited to the patient's virtual room when it is an ‘exam room.’ As another example, Dr. Tuesday may be a room participant because the physician's medical practice has an appointment scheduled for Todd Monday with Dr. Tuesday at 10:10 am and the access rule indicates the physician scheduled is added to the exam room. One or more other room participants may be invited, either via application of access rules or because they are invited by Dr. Tuesday via selection of new participant control 730. For example, a nurse may be invited to the virtual room because it is an ‘exam room’ and the room access rules for the physician's office indicate a participant with a nurse role should be invited. As another example, Dr. Tuesday may invite a dietary specialist to the virtual room via activation of control 730. Activation of new participant control 730 enables Dr. Tuesday to enter a phone number or email address for the specialist so the specialist is invited as a participant.

The example user interface 700 may include a main viewing area 740. The main viewing area 740 includes an event history 715 area. The event history 715 area may list some or all of the events recorded in the event history for the visit, e.g., in event history 235a. In the example of FIG. 7, the event history 715 is similar to the event history 515 area of FIG. 5, but includes additional events, such as the change in room status resulting from successful completion of the video capability test. The main viewing area 740 of the example user interface 700 may include a messaging area 720. The messaging area 720 may enable room participants to send text communications, files, images, etc. within the virtual room, as discussed with regard to messaging area 520 of FIG. 5. The messages may appear in the main viewing area 740, for example as messages 750. The main viewing area 740 may have a separate area for messages 750 or may include messages 750 interspersed with the event history 715 (e.g., organized by time).

The example user interface 700 also includes a virtual visit data panel 725. The virtual visit data panel 725 operates in a similar manner to the virtual visit data panel 525 of FIG. 5. But in the example of FIG. 7 the virtual visit data panel 725 relates to team participants, or in other words, those who are invited to a subject-centered chat. Thus, for example, if Dr. Tuesday invites additional team members to the virtual room (or if a nurse were invited due to application of room access rules), information identifying the team participants would be displayed in the virtual visit data panel 725. The virtual visit data panel 725 for chat members may also include messaging area 755. Messaging area 755 may send messages to the subject-centered chat, which are displayed for team participants and not to room participants that are not also team participants. In some implementations the messages in the subject-centered chat may be displayed in virtual visit data panel 725. In some implementations, the messages in the subject-centered chat may be displayed in the main viewing area 740, but only for team participants.

The data and controls displayed as part of the user interface 700 may depend on the room status and the participant role. For example, because the virtual visit object displayed in the main viewing area 740 has an ‘exam room’ status and the user has a physician role, the main viewing area 740 may include one or more status messages 735. The status messages 735 may relate to an event (e.g., the subject participant is ready to participate in a video call) and include additional information, in this case a control 745 that enables the physician to start a video conference. The control 745 enables the physician to control when the video conference starts, in the exam room, to ensure that the conference does not begin until the participant with the physician role is ready. In some implementations, all or part of a status message may be included in the event history 715.

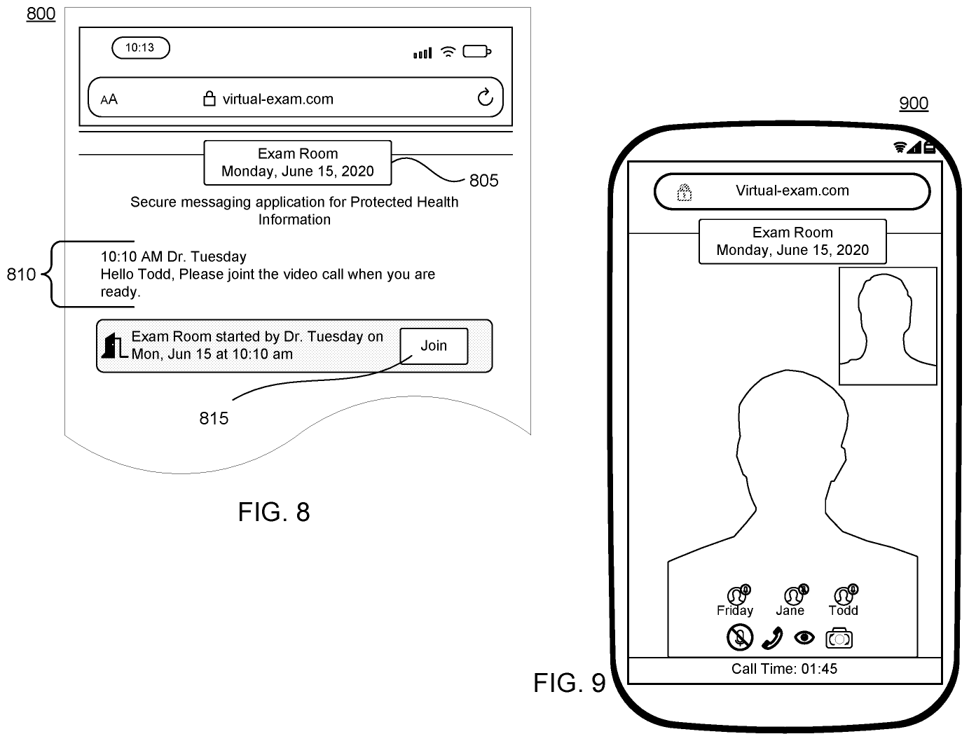

FIG. 8 illustrates an example user interface 800 for a patient in a virtual room with an ‘exam room’ status, according to a disclosed implementation. In the example of FIG. 8, the subject participant (Todd Monday) has successfully completed the video capability test control, which causes the room status of the virtual room to change from ‘waiting room’ to ‘exam room’. With this change in status, the system may automatically change the room participants, as described above. The example user interface 800 may include an indication of the room status 805. The indication of the room status 805 can be text based or a visual cue (color, icon, etc.), or a combination of these. The example user interface 800 may display messages 810 sent to room participants. Although not illustrated, the example user interface 800 may include a messaging area, similar to messaging area 620 of FIG. 6, that enables the participant to add to the messages 810.

The example user interface 800 may also include a control 815 that enables the participant to join a video call, once the call has been initiated by the physician or other participant with the permissions to start such a call.

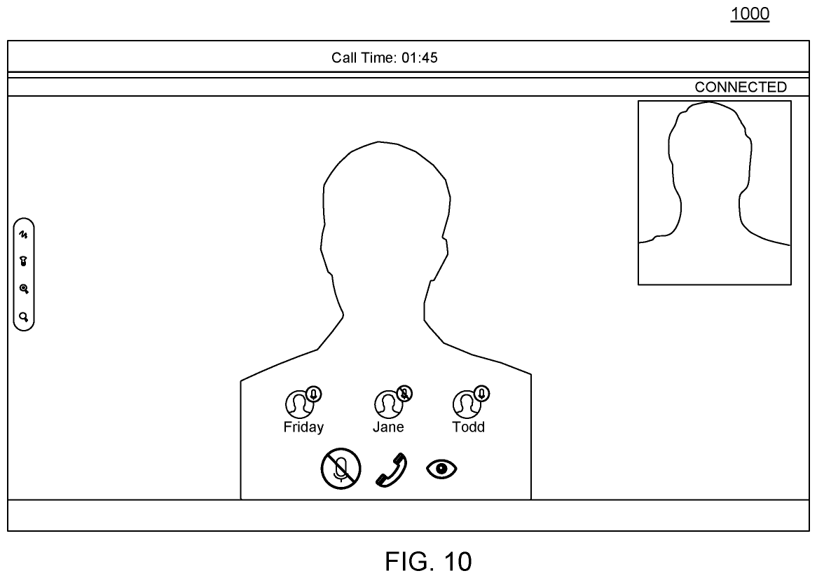

FIG. 9 illustrates an example user interface 900 for a secure video session that occurs in the virtual room with an ‘exam room’ status, according to a disclosed implementation. The example user interface 900 is for a mobile device and may be an example of a user interface provided to a patient or service team participant during video communications in a virtual room. The video communications may be secure video communications using known or later developed techniques. FIG. 10 illustrates an example user interface 1000 for a secure video session that occurs in the virtual room with an ‘exam room’ status, according to a disclosed implementation. The example user interface 1000 is for a desktop device and may be an example of a user interface provided to a service team participant during video communications in a virtual room. In the example of FIGS. 9 and 10 the subject participant has added a room participant (Jane) and Dr. Tuesday has added a participant (Specialist Friday) to the exam room during the video call.

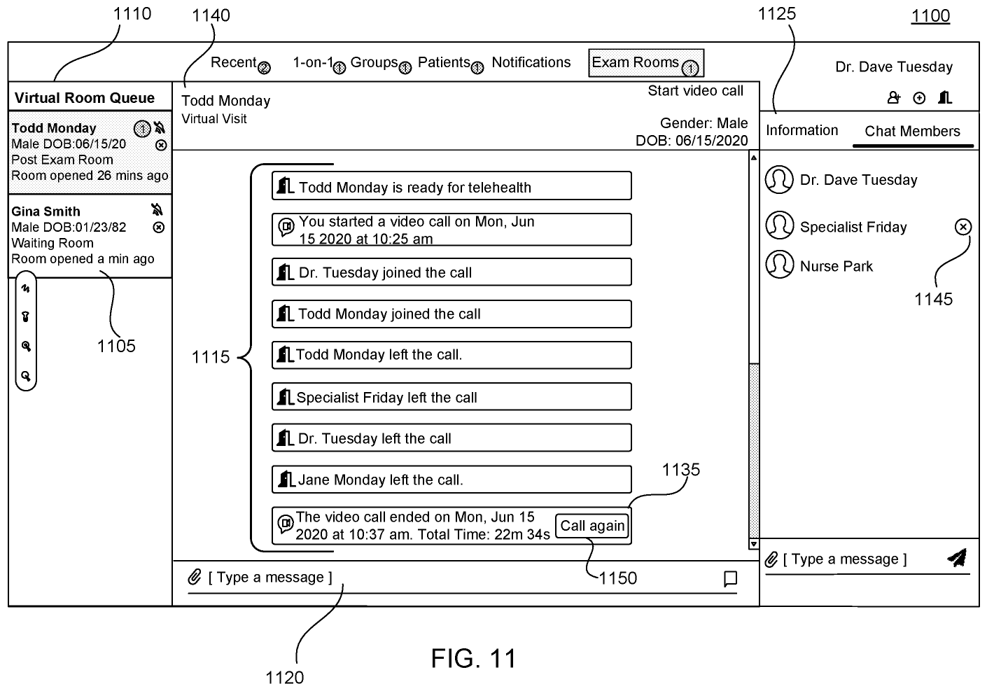

FIG. 11 illustrates an example user interface 1100 for a physician in the virtual room with a ‘post exam room’ status, according to a disclosed implementation. In the example of FIG. 11, the video call has ended, which in this example may result in a change of room status, e.g., from ‘exam room’ to ‘post exam room’. With this change in status, the system may automatically change the room participants, depending on the applicable virtual visit object access rules. In the example of FIG. 11, the change in status results in Nurse Park being added to the room participants and to the team participants. In some implementations, participants added to the room participants by another participant may be removed when the room status changes. Put another way, some participant's access to a virtual visit object may be revoked when the room status changes. In some implementations, the participants added to the virtual room by other participants may continue as room participants after a change in room status. For example, Specialist Friday may remain a room participant until Dr. Tuesday explicitly removes her, e.g., via control 1145 in the virtual visit data panel 1125. In some implementations, whether a participant added by another participant remains a room participant after a room status change may be determined by one or more virtual room access rules applicable to the new room status.

The example user interface 1100 includes a virtual room queue 1110, which operates similarly to the virtual room queue 710 of FIGS. 7 and 510 of FIG. 5. In the example of FIG. 11, the virtual room queue 1110 shows another virtual visit object summary 1105, which has a virtual room with a ‘waiting room’ status. The virtual visit object summary 1105 appears in the virtual room queue 1110 because Dr. Tuesday is a room participant and/or a team participant for the virtual visit object for Gina Smith. The example user interface 1100 is configured to change the content of the main viewing area 1140 and the virtual visit data panel 1125 to display information related to the virtual visit object for Gina Smith responsive to selection of the virtual visit object summary 1105.

The main viewing area 1140 in the example user interface 1100 includes event history 1115 area. The event history 1115 area provides information about what has occurred during the virtual visit and may be a scrollable list of room events. Thus, for example, the event history 1115 area is a continuation of the event history 715 area of FIG. 7. The event list includes at least one status message 1135, which combines the event with a control 1150. The control 1150 is configured to be selected by the participant. Selection of the control 1150 may change the room status back to ‘exam room’ and initiate a second video conference. The inclusion of the control 1150 may be role-based. Thus, for example, a participant in a physician role may have the control 1150 included while a participant with the nurse role or a participant invited by the physician may not see the control 1150.

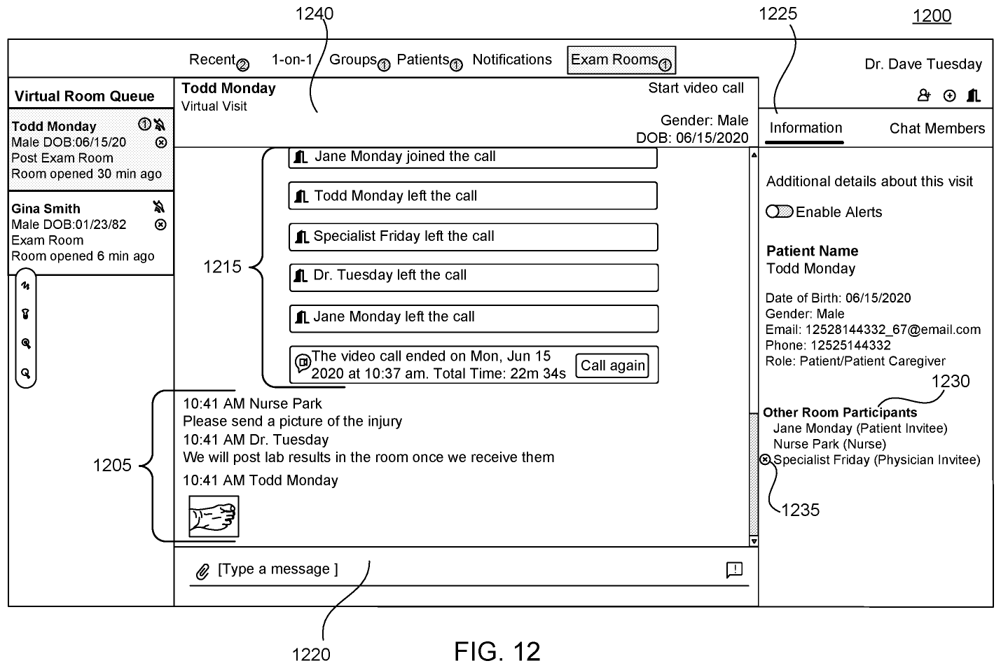

FIG. 12 illustrates an example user interface 1200 for a physician in the virtual room with a ‘post exam room’ status, according to a disclosed implementation. The example of FIG. 12 illustrates the example user interface 1100 at a later point in time. Thus, for example, the main viewing area 1240 includes text messages 1205 sent in the room-by-room participants. The messages may have been entered via the messaging area 1120 or 1220. In some implementations, the text messages 1205 may be available while the room status is ‘post exam room’ but may not be viewable in the main viewing area 1240 if the room status changes. Thus, for example, the text messages 750 of FIG. 7 may not be viewable in the event history 1215 by scrolling up. In some implementations, the text messages 1205 may be viewable in the event history 1215. Thus, for example, the text messages 750 of FIG. 7 may be viewable in the scrollable event history 1215 by scrolling up.

The example user interface 1200 also illustrates a virtual visit data panel 1225 showing information about the virtual visit object. In this example, the information includes room participants 1230. The list of room participants 1230 includes participants other than the subject participant and the participant for which example user interface 1200 is generated. In some implementations, the list of room participants 1230 may include the participant role. In some implementations. In some implementations, the example user interface may enable a participant to remove an invitee from the room. Thus, for example, Dr. Tuesday may remove Specialist Friday, e.g., via control 1235.

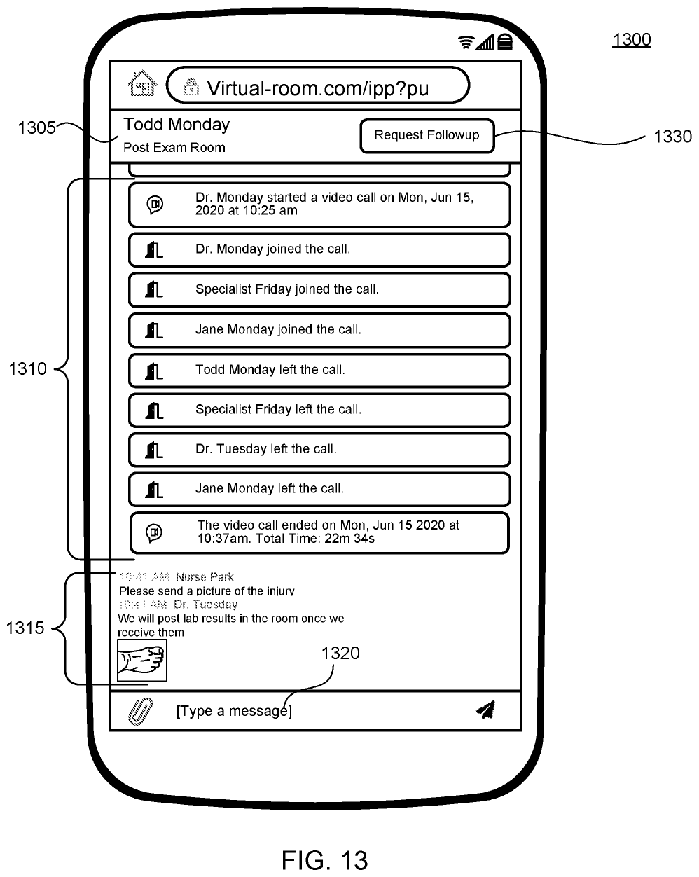

FIG. 13 illustrates an example user interface 1300 for a patient in the virtual room with a ‘post exam room’ status, according to a disclosed implementation. The example user interface 1300 roughly corresponds to the example user interface 1200 of FIG. 12, but for a different role (the subject participant rather than the physician participant) and for a different platform (mobile instead of desktop). Thus, example user interface 1300 represents a virtual room interface configured for room status (e.g., post exam room) and participant role (e.g., subject participant) while the example user interface 1200 of FIG. 12 represents the virtual room interface configured for room status (e.g., post exam room) and participant role (e.g., physician).

The example user interface 1300 includes a room status indicator 1305 and an event history 1310 list, similar to the event history 1215 list. The subject patient can participate in the room chat by using messaging area 1320. The example user interface 1300 includes text messages 1315 sent to the room chat, as explained with regard to text messages 1205. The example user interface 1300 may lack the control 1150 that was available to the physician role, but may include control 1330, which may enable the subject participant to request a follow-up call.

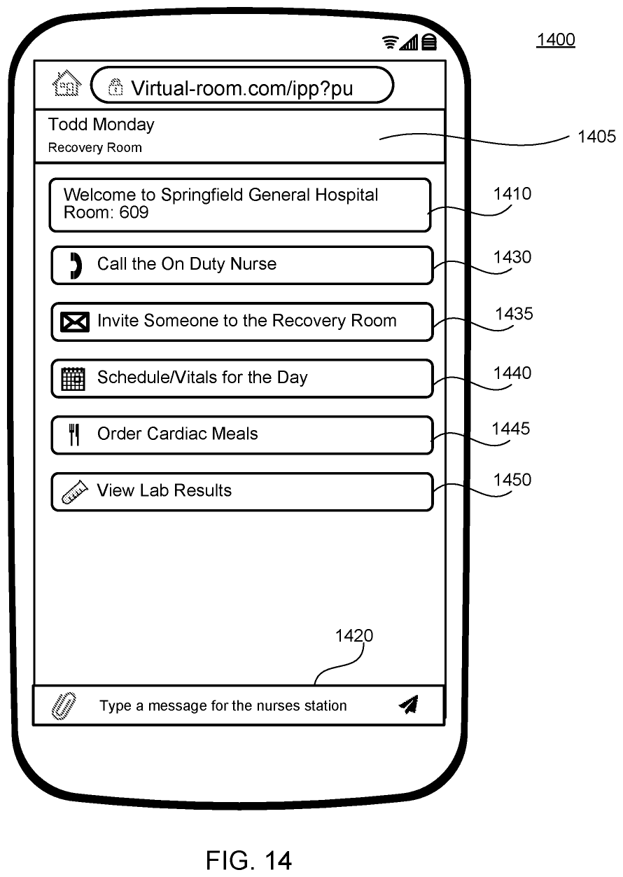

FIG. 14 illustrates an example user interface 1400 for a patient in the virtual room with a ‘recovery room’ status subsequent to the secure video session, according to a disclosed implementation. In the example of FIG. 14, the subject patient has been admitted to a hospital and is currently in a recovery room of the hospital. Although physically present in the hospital, the virtual visit object enables the patient to have access to particular services and also facilitates video or telephone consultations with participants who cannot be present at the hospital. For example, during the COVID-19 pandemic hospitals have restricted access to hospital facilities, meaning that family members cannot be present for consultations and do not have access to vitals, schedules, etc. for their family member and often feel disconnected. The virtual visit object encapsulates the information and provides a platform (including user interfaces) to provide access to this information that was not previously available. The example user interface 1400 includes a room status indicator 1405, which indicates the current virtual room status is ‘recovery room.’ Based on this status and the participant role, the example user interface 1400 includes other information, such as the location 1410 of the physical recovery room.

The example user interface 1400 may also include control 1430. Control 1430 may initiate a user interface that displays information about scheduled assignments. For example, a patient may be able to see who is currently assigned to the virtual room in the role of nurse and when (at what time) a second person will be assigned, e.g., based on an upcoming schedule change. The example user interface 1400 may also include control 1435. Control 1435 enables the patient to invite others (e.g., family, friends, etc.) to the virtual room. As a room participant, the invitee may have access to all the data elements associated with the virtual visit object or only some of the data elements associated with the virtual visit object. What data elements are available may be determined by the room status and the role (e.g., subject invitee). In some implementations the subject invitee may have access to the event history for the virtual visit object. In some implementations, a family member may be invited to the virtual room by a team member. For example, if the patient is in an ICU room at the hospital (e.g., and the room status is ‘ICU room’) a member of the service team may invite a family member of the patient to the virtual room. Thus, the family member may be able to see the schedule, vitals, lab results, etc. to keep track of the patient without having to wait on calls from hospital staff. Moreover, from within the room a physician or other team member may be able to schedule a video call with the family member easily, without having to look up or exchange contact information.

Example user interface 1400 also includes control 1445, which may display a particular dietary menu for the patient when selected. Thus, for example, the virtual room may account for dietary restrictions placed on the patient and may limit dietary options to those in compliance with the restrictions. This may be done via a combination of information known about the subject participant and data sources/APIs available via the hospital records system. The control 1445 may result in a user interface that lists acceptable meal options for cardiac patients and enables the subject participant to order options for delivery to the room.

Example user interface 1400 also includes control 1440. Control 1440 may initiate display of a user interface that shows a schedule, e.g., timing of tests, specialist visits, medication, etc., for the subject participant. In some implementations, the scheduling of a test may be an event in the event history for the virtual visit object. In some implementations, the interface initiated by selection of control 1440 may also display vitals and other information about the patient. This information is often tracked on a white board in the patient's room, but using disclosed systems such information could be recorded in the virtual visit object (e.g., as part of collected data), and available for viewing by room participants and/or team participants. This would give room invitees invited by the patient access to this information from home and enables team participants to track the information as well without having to be present in the room. Such information, as part of the virtual visit object, expires when the virtual visit object expires and does not become part of the patient's permanent medical record.

Example user interface 1400 may also include control 1450. Selection of control 1450 may provide a user interface that displays lab results or other information maintained by the hospital for the subject patient. This information may be available using APIs provided by the hospital. Other information not specifically illustrated in example user interface 1400 may also be made available to the patient.

The example user interface 1400 includes messaging area 1420. The subject patient can participate in the room chat by using messaging area 1420. The room chat is accessible by room participants. In this example, an account with the role of nurse's station attendant may be a room participant and/or the on-duty nurse for the floor may be a room participant. An attending physician may not be a room participant, but may be a team participant. Thus, implementations may enable a patient to contact an on-duty nurse via messaging area 1420 and the on-duty nurse may be able to contact an attending physician via a subject-centered chat provided within the virtual visit object. Thus, implementations encapsulate communications around the subject participant, with participants automatically determined based on the subject participant, current purpose of the visit, and applicable access rules.

The following examples illustrate how the novel infrastructure described herein provides a specific application of technical tools to provide organization, data collection, facilitate timely communication, and automate and improve continuity of care. In an example scenario, a virtual visit object may be generated in response to a patient request for a telemedicine visit. The initial room status may be ‘waiting room,’ e.g., where the patient may be requested to fill out a form. The form may be associated with virtual rooms having a ‘waiting room status’ in the enterprise (room access) rules. The team participants for such a room status may be a receptionist and/or a nurse at the physician's office. The patient may chat with the receptionist while the virtual room has the “waiting room” status. A physician may have a queue of virtual room requests and may be able to see events for each of these requests, e.g., see which ones have completed intake forms and/or teleconference readiness tasks, as these events may be recorded in the event history. The physician may select the patient's virtual visit object, and this may change the virtual room status to “exam room”. This status may invite additional team members (e.g., an interpreter) and uninvite the receptionist or nurse. Changing the virtual room status to ‘exam room’ may also activate a teleconference with the physician. Any participant the patient has invited may participate in the teleconference (e.g., family member, interpreter, etc.). The event history may record who participated in the teleconference and when the teleconference took place (starting time and duration). The physician may end the call, changing the virtual room status to ‘post-exam room’, which may invite the nurse back to the virtual room. The nurse can wrap up the visit, ending the last service session (ending the episode of medical care). The virtual visit object may exist for some period of time after the end of the last service session.

In another example scenario, a patient may arrive at a hospital with a behavioral health issue. A virtual visit object may be created when the patient is admitted. At some point after admission, the virtual room may have a status of ‘behavior exam room’, which may invite a behavioral health specialist to the team participants. During a teleconference in the virtual room, the behavioral health specialist may invite a nutritionist. Once the nutritionist has joined, the behavioral health specialist may leave. When the teleconference ends, the nutritionist (or another attending physician) may change the virtual room status, e.g., to ‘observation room’ or something similar. The details of the status changes, participants, teleconferences, forms filled out, etc., are recorded in the event history, so that as team members are invited to the virtual room information regarding continuity of care is readily available in an organized fashion. The visit object may be closed upon discharge of the patient but may be deleted some period of time after discharge.

In another example scenario, a company's recruiter may arrange a virtual job interview. The recruiter may send an invitation at an agreed upon time to a job candidate (the subject participant) and a virtual visit object generated for the candidate. After accepting the invitation, the candidate may be presented with an interface for a ‘reception room’ where the recruiter and the candidate are the room participants. A room status change to ‘conference room 1’ may change the room participants to the job candidate, a first employee and a second employee. A video conference may take place in the virtual room between the room participants. At the close of the video conference the room status may change to ‘conference room 2’. This change in status may change the room participants to two different employees. All four employees may be considered team members and may use subject-centered chat. A change in status to ‘wrap up room.’ may change the room participants back to the job candidate and the receptionist where the job candidate can use the room chat for follow-up questions. In some implementations, the interview visit may be partially virtual, e.g., one of the employees may join virtually but the job candidate and another employee may be meeting in person. This still enables the team participants to make use of the subject-centered chat.

In addition to the configurations described above, an apparatus can include one or more apparatuses in computer network communication with each other or other devices. In addition, a computer processor can refer to one or more computer processors in one or more apparatuses or any combinations of one or more computer processors and/or apparatuses. An aspect of an implementation relates to causing and/or configuring one or more apparatuses and/or computer processors to execute the described operations. The results produced can be output to an output device, for example, displayed on the display. An apparatus or device refers to a physical machine that performs operations, for example, a computer (physical computing hardware or machinery) that implement or execute instructions, for example, execute instructions by way of software, which is code executed by computing hardware including a programmable chip (chipset, computer processor, electronic component), and/or implement instructions by way of computing hardware (e.g., in circuitry, electronic components in integrated circuits, etc.)—collectively referred to as hardware processor(s), to achieve the functions or operations being described. The functions of implementations described can be implemented in any type of apparatus that can execute instructions or code.

More particularly, programming or configuring or causing an apparatus or device, for example, a computer, to execute the described functions of implementations creates a new machine where in case of a computer a general-purpose computer in effect becomes a special purpose computer once it is programmed or configured or caused to perform particular functions of the implementations pursuant to instructions from program software. According to an aspect of an implementation, configuring an apparatus, device, computer processor, refers to such apparatus, device or computer processor programmed or controlled by software to execute the described functions.

A program/software implementing the implementations may be recorded on a computer-readable media, e.g., a non-transitory or persistent computer-readable medium. Examples of the non-transitory computer-readable media include a magnetic recording apparatus, an optical disk, a magneto-optical disk, and/or volatile and/or non-volatile semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), DVD-ROM, DVD-RAM (DVD-Random Access Memory), BD (Blue-ray Disk), a CD-ROM (Compact Disc-Read Only Memory), and a CD-R (Recordable)/RW. The program/software implementing the implementations may be transmitted over a transmission communication path, e.g., a wire and/or a wireless network implemented via hardware. An example of communication media via which the program/software may be sent includes, for example, a carrier-wave signal.

The many features and advantages of the implementations are apparent from the detailed specification and, thus, it is intended by the appended claims to cover all such features and advantages of the implementations that fall within the true spirit and scope thereof. Further, since numerous modifications and changes will readily occur to those skilled in the art, it is not desired to limit the inventive implementations to the exact construction and operation illustrated and described, and accordingly all suitable modifications and equivalents may be resorted to, falling within the scope thereof.

Those skilled in the art will recognize that the present teachings are amenable to a variety of modifications and/or enhancements. For example, the automatic anonymization system and its components as disclosed herein can be implemented as a firmware, firmware/software combination, firmware/hardware combination, or a hardware/firmware/software combination.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

In one general aspect, a system includes at least one processor, a virtual visit object data store storing virtual visit objects and memory storing instructions that, when executed by the at least one processor, causes the system to perform operations. Each virtual visit object may be generated for a respective subject participant and including a virtual room, and event history records, the virtual room having a room status and room participants determined at least by an access rule applicable to the room status of the virtual room. The operations may include generating a first virtual visit object in the virtual visit object data store, the first virtual visit object including a virtual room with a first room status and inviting room participants to join the virtual room, the room participants being determined at least by a first access rule for the first room status, the room participants including a subject participant. The operations may also include providing access to the virtual room to invited room participants who accept, the access including a first virtual room interface configured for the first room status based on room participant role, wherein providing access includes recording room events in the event history records for the first virtual visit object. The operations may further include providing access to a subject-centered communication channel based on team access rules for the first room status, the subject-centered communication channel lacking the subject participant and receiving a room status change for the virtual room to a second room status. Responsive to the room status change, the operations may also include recording the room status change in the event history records for the first virtual visit object, updating the room participants according to at least a second access rule for the second room status, including adding new room participants based on roles identified in the second access rule and revoking access from room participants with roles not identified in the second access rule, providing access to the virtual room to the room participants who accept, the access including a second virtual room interface configured for the second room status based on room participant role, and providing access to the subject-centered communication channel based on team access rules for the second room status.

These and other aspects may include one or more of the following features, alone or in combination. For example, the system may also include an access rules data store for storing at least some of the access rules, the access rules including room access rules, each room access rule identifying, for a particular room status, team participants by role and room participants by role. In some implementations, the access rules may also include room participant access rules, each room participant access rule identifying, for a particular room status, data items to be collected or data items to be presented in a virtual room interface configured for the particular room status. As another example, providing access to the virtual room includes determining a data item for collection in the virtual room based on the first room status, collecting the data item via the first virtual room interface, and storing the data item as part of the first virtual visit object. As another example, the first virtual room interface may enable the subject participant to invite another participant to the virtual room when the virtual room has the first room status and/or the second virtual room interface may enable video communications between the subject participant and other room participants. In some implementations, the room events include identifying a timestamp for a beginning of the video communications and a timestamp for an end of the video communications. As another example, the room events may include identifying room participants that join the virtual room and identifying room participants that leave the virtual room. As another example, the operations may also include deleting the first virtual visit object from the virtual visit object data store for a virtual visit older than a predetermined time period. As another example, the first virtual visit object may be for a medical visit and the first room status is a waiting room status and the second room status is an exam room status. In some implementations, the first access rule identifies a plurality of data elements collected via the first virtual room interface for the subject participant and wherein one of the room participants has a receptionist role. In some further implementations, the room status changes from the first room status to the second room status responsive to the subject participant submitting the plurality of data elements and completing a video requirements test and/or at least one of the plurality of data elements collected is displayed in the second virtual room interface.

According to one aspect, a method comprises receiving a request for a virtual visit from a patient and instantiating a virtual visit object for the patient including assigning a room status of a virtual room in the virtual visit object to a first room status, assigning a first service team member to room participants for the virtual room based on a role identified in a first room access rule applicable to the first room status, and assigning the patient to a subject participant role and sending an invitation to the virtual room to the patient. The method may also include providing a first user interface to the patient responsive to the patient accepting the invitation, the first user interface being configured according to room participant access rules for the first room status, the room participant access rules including a rule for collecting a medical record data element. The method may further include obtaining the medical record data element for the patient via the first user interface, changing the room status to a second room status, assigning a second service team member to the room participants for the virtual room based on a role identified in a second room access rule applicable to the second room status, removing the first service team member from the room participants responsive to the second room access rule lacking the role for the first service team member, and providing a second user interface to the patient, the second user interface being configured according to access rules for the second room status.

These and other aspects can include one or more of the following, alone or in combination. For example, the method may also include providing a virtual room queue to the second service team member, the virtual room queue listing virtual visit objects with virtual rooms for which the second service team member is a room participant. As another example, the method may include assigning a third service team member as a team participant for a subject-centered chat associated with the virtual visit object based on an invitation from the second service team member, wherein communications occurring in the subject-centered chat are tied to the virtual visit object and not to team participants. As another example, the second user interface includes a video conference interface and the method may also include providing a secure video conference via the second user interface and storing timestamps for when room participants joined the virtual room, timestamps for when room participants left the virtual room, a timestamp for the room status change, and timestamps for a beginning and an end of the secure video conference in an event history associated with the virtual visit object. In some implementations, the first user interface includes a video capabilities test and changing the room status occurs after a successful test. As another example, the method can include transferring the medical record data element to a medical records system to store as part of a record for the patient. As another example, the method may also include assigning a third service team member as a room participant based on a role identified in the second room access rule applicable to the second room status and a schedule. As another example, the method may also include providing access to subject-centered chat communications associated with the virtual visit object to team participants, wherein at least some of the team participants change with a room status change and at least some of the team participants change with a schedule change.

In one general aspect, a system for coupling a virtual examination room with a patient center chat group to create virtual visit objects. In another aspect, a computer program product embodied on a computer-readable storage device includes instructions that, when executed by at least one processor formed in a substrate, cause a computing device to perform any of the disclosed methods, operations, or processes disclosed herein.

1. A system comprising:
at least one processor;
a virtual visit object data store storing virtual visit objects, each virtual visit object being generated for a respective subject participant and including a virtual room, and event history records, the virtual room having a room status and room participants determined at least by an access rule applicable to the room status of the virtual room; and
memory storing instructions that, when executed by the at least one processor, causes the system to perform operations including:
generating a first virtual visit object in the virtual visit object data store, the first virtual visit object being generated for a subject participant and including a virtual room with a first room status,
determining room participants for the virtual room, the room participants being determined at least by a first access rule for the first room status, the room participants including the subject participant,
inviting the room participants to join the virtual room,
providing access to the virtual room to invited room participants who accept, the access including a first virtual room interface configured for the first room status based on room participant role, wherein providing access includes recording room events in the event history records for the first virtual visit object,
providing access to a subject-centered communication channel based on team access rules for the first room status, the subject-centered communication channel lacking the subject participant,
receiving a room status change for the virtual room to a second room status, and
responsive to the room status change:
recording the room status change in the event history records for the first virtual visit object,
updating the room participants according to at least a second access rule for the second room status, including adding new room participants based on roles identified in the second access rule and revoking access from room participants with roles not identified in the second access rule,
providing access to the virtual room to the room participants who accept, the access including a second virtual room interface configured for the second room status based on room participant role, and
providing access to the subject-centered communication channel based on team access rules for the second room status.

2. The system of claim 1, further comprising an access rules data store for storing at least some of the access rules, the access rules including the team access rules and further including:
room access rules, each room access rule identifying, for a particular room status, team room participants by role,
team participant access rules, each team participant access rule identifying, for a particular room status, data items to be presented in a virtual room interface configured for the particular room status, and
room participant access rules, each room participant access rule identifying, for a particular room status, data items to be collected or data items to be presented in a virtual room interface configured for the particular room status.

3. The system of claim 1, wherein providing access to the virtual room includes:
determining a data item for collection in the virtual room based on the first room status;
collecting the data item via the first virtual room interface; and
storing the data item as part of the first virtual visit object.

4. The system of claim 1, wherein the first virtual room interface enables the subject participant to invite another participant to the virtual room when the virtual room has the first room status.

5. The system of claim 1, wherein the second virtual room interface enables video communications between the subject participant and other room participants.

6. The system of claim 5, wherein the room events include identifying a timestamp for a beginning of the video communications and a timestamp for an end of the video communications.

7. The system of claim 1, wherein the room events include identifying room participants that join the virtual room and identifying room participants that leave the virtual room.

8. The system of claim 1, wherein the operations further include deleting the first virtual visit object from the virtual visit object data store for a virtual visit older than a predetermined time period.

9. The system of claim 1, wherein the first virtual visit object is for a medical visit and the first room status is a waiting room status and the second room status is an exam room status.

10. The system of claim 9, wherein the first access rule identifies a plurality of data elements collected via the first virtual room interface for the subject participant and wherein one of the room participants has a receptionist role.

11. The system of claim 10, wherein the room status changes from the first room status to the second room status responsive to the subject participant submitting the plurality of data elements and completing a video requirements test.

12. The system of claim 10, wherein at least one of the plurality of data elements collected is displayed in the second virtual room interface.

13. A method comprising:
receiving a request for a virtual visit from a patient;
responsive to the request, instantiating a virtual visit object for the patient including:
assigning a room status of a virtual room in the virtual visit object to a first room status,
assigning a first service team member to room participants for the virtual room based on a role identified in a first room access rule applicable to the first room status, and
assigning the patient to a subject participant role and sending an invitation to the virtual room to the patient
wherein the virtual visit object provides a subject-centered communication channel based on team access rules for the first room status, the subject-centered communication channel lacking the patient;
providing a first user interface to the patient responsive to the patient accepting the invitation, the first user interface being configured according to room participant access rules for the first room status, the room participant access rules including a rule for collecting a medical record data element;
obtaining the medical record data element for the patient via the first user interface;
changing the room status to a second room status;
assigning a second service team member to the room participants for the virtual room based on a role identified in a second room access rule applicable to the second room status;
removing the first service team member from the room participants responsive to determining the second room status lacks an access rule for the role for the first service team member; and
providing a second user interface to the patient, the second user interface being configured according to access rules for the second room status.

14. The method of claim 13, further comprising:
providing a virtual room queue to the second service team member, the virtual room queue listing virtual visit objects with virtual rooms for which the second service team member is a room participant.

15. The method of claim 13, further comprising:
assigning a third service team member as a team participant for the subject-centered communication channel based on an invitation from the second service team member,
wherein communications occurring in the subject-centered communication channel are tied to the virtual visit object and not to team participants.

16. (canceled)

17. (canceled)

18. The method of claim 13, wherein the method includes transferring the medical record data element to a medical records system to store as part of a record for the patient.

19. The method of claim 13, further comprising:
assigning a third service team member as a room participant based on a role identified in the second room access rule applicable to the second room status and a schedule.

20. The method of claim 13, further comprising:
providing access to subject-centered chat communications associated with the virtual visit object to team participants,
wherein at least some of the team participants change with a room status change and at least some of the team participants change with a schedule change.

21. The method of claim 13, wherein the virtual visit object for the patient is stored in a virtual visit object data store, each virtual visit object in the data store including:
subject-centered chat data;
current team participants;
current room participants; and
event history data.

22. The method of claim 21, further including deleting the virtual visit object from the virtual visit object data store after a predetermined time period.
